(19) United States
(12) Patent Application Publication
(10) Pub. No.: US 2012/0004112 A1
(43) Pub. Date: Jan. 5, 2012

(54) METHODS FOR DETERMINING A BREEDING VALUE BASED ON A PLURALITY OF GENETIC MARKERS

(75) Inventors: Mogens Sandø Lund, Orum (DK); Guosheng Su, Viborg (DK); Bernt Guldbrandtsen, Tjele (DK)

(73) Assignees: AARHUS UNIVERSITET, Arhus C (DK); VIKING GENETICS FMBA, Randers SO (DK)

(21) Appl. No.: 13/059,455

(22) PCT Filed: Aug. 19, 2009
(86) PCT No.: PCT/DK2009/050203

§ 371 Date: Sep. 26, 2011

(30) Foreign Application Priority Data
Aug. 19, 2008 (DK) PA 2008 01128

(51) Int. Cl. C40B 20/00; C12Q 1/68; C40B 30/04

(52) U.S. Cl. 506/2; 506/9; 435/6.11

(57) ABSTRACT

The present invention provides a method for determining the individual effect of a plurality of genetic marker alleles on udder health, fertility and/or other health of a plurality of reference bovine subjects. The marker effects are employed in another aspect of the invention for determining a genomic estimated breeding value of a bovine subject based on the genotype of said bovine subject by correlating its genotype with the effect of each individual genetic marker allele on udder health, fertility, other health and/or estimated breeding value of the reference bovine subjects. Further provided are methods and computer program products and computer readable media for executing the methods of the invention.

### FIELD OF INVENTION

The present invention relates to methods and products for estimation on a breeding value based in genomic information. In one aspect, the invention relates to a method for determining the individual effect of a plurality of genetic markers on a phenotype such as udder health, fertility, or other health, or breeding value of reference bovine subjects. The individual effects of the genetic markers are employed in another aspect of the invention, for estimating a breeding value of a bovine subject based on the genotype of said bovine subject for the plurality of genetic markers.

### BACKGROUND OF INVENTION

The identification of genetic markers, that are associated with a particular phenotype, such as quantitative traits or to a heritable disease, has been facilitated by the identification of an increasing amount of markers such as microsatellite markers and single nucleotide polymorphisms (SNPs) as a source of polymorphic markers, which are associated with a mutation causing a specific phenotype. Markers associated with the mutation or the mutation itself causing a specific phenotype of interest may be localised by use of genetic analysis in pedigrees. In fact, application of molecular genetic information is an important issue in animal breeding.

Conventional methods to predict breeding value of cattle is based on the phenotypic record of the individual and the records of its relatives. Thus, the estimation of a breeding value of a bovine subject requires that phenotypic traits of the bovine subject and/or its relatives have been registered. Other methods of estimating a breeding value combine phenotypic and pedigree data with genomic information to increase accuracy and decrease generation interval. In contrast to other genetic evaluation methods, which only combine phenotypic data and probabilities that genes are identical by descent from pedigree data, genomic prediction traces the inheritance of individual genes. Thus, marker genotypes for multiple genetic marker loci across the genome can measure genetic similarity with higher precision than single marker methodologies and marker assisted selection based on the inheritance of only a few major genes. Low resolution marker maps can only provide indications of shared long chromosome segments within closely related family members but cannot detect the many minor genetic effects shared by distant relatives. In contrast by using genomic prediction, even minor genes can now be traced using high density genetic markers located across the entire genome. Current reports on genomic selection are primarily based on simulated data. Recently, a few results based on the data from real livestock population have been published (e.g., Harris et al., 2008; Hayes et al., 2009; Gonzalez-Recio et al., 2009; VanRaden et al., 2009). None of these reports, however, evaluate the accuracy of genomic prediction in the target population, which is necessary in order to apply genomic selection in practical breeding programs.

### SUMMARY OF INVENTION

In a key aspect, the present invention relates to a method for determining the individual effect of a plurality of genetic markers on a breeding value of one or more reference bovine subjects. The invention also relates to estimation of a breeding value in a bovine subject, wherein said breeding value is based in the genotype of the bovine subject for a plurality of genetic markers. The estimated breeding value is then determined on the basis of the individual effect of the plurality of genetic markers on the breeding value of a reference bovine subjects. Moreover, the present invention relates to a method for selective breeding based in the estimation of the genomic breeding value; and the invention also comprises computer program products, computer readable media and computer systems for executing the methods of the present invention. A kit is also provided, which comprises one or more components of the present invention.

Also, in a main object of the present invention to provide an application method for marker assisted selection of polymorphisms in the bovine genome, wherein polymorphisms are associated with specific traits and phenotypes, such as defined herein; and/or provide a kit for detection of genetic marker alleles or a combination of genetic marker alleles for use in such a method, and/or to provide animals selected using the method of the invention.

Thus, in one aspect, the present invention relates to a method for determining the individual effect of a plurality of genetic marker alleles on udder health, fertility and/or other health of at least 100 reference bovine subjects and/or its relatives, said method comprising

a. providing at least 100 reference bovine subjects,
b. obtaining a sample from said one or more bovine subjects comprising genetic material,
c. determining on the basis of said genetic material the genotype of said one or more reference bovine subjects for said plurality of genetic markers,
d. determining the udder health, fertility and/or other health of said reference bovine subject, and
e. determining the individual effect of said plurality of genetic marker alleles on said udder health, fertility and/or other health of said reference bovine subject.

In a preferred embodiment, an estimated breeding value (EBV) is calculated on the basis of said udder health, fertility and/or other health. For example, the effect of each individual genetic marker allele on udder health, fertility, and/or other health is determined by calculating a reference estimated breeding value (EBV) of said one or more reference bovine subjects, wherein said reference EBV is used as response variable for determining the effect of each individual genetic marker allele on udder health, fertility, other health and/or the EBV. Udder health, fertility, other health and/or estimated breeding value is preferably determined by registration of phenotypic traits of said bovine subject and off-spring and/or other relatives of said bovine subject.

Udder health, fertility, other health and/or estimated breeding value is determined according to any method available to those of skill in the art. The phenotypes are preferably evaluated by registration of phenotypic traits of at least 40 offspring or other relatives of said bovine subject, however, the more offspring or relatives scored for a specific phenotype, the more accurate is the determination of the phenotype. Thus, in one embodiment, the effect of the plurality of genetic markers on udder health, fertility, other health and/or estimated breeding value have been determined for at least 100 reference bovine subjects, such as at least 1000 bovine subjects, for example between 1000 and 6000 bovine subjects, such as between 2000 and 5000 bovine subjects. In some cases at least 10000, such as for example between 10000 and 50000 bovine subjects (e.g. offspring or relatives) are included in the determination of a phenotype and/or a reference estimated breeding value.

Udder health, fertility, other health and/or estimated breeding value is in one embo determined using Least-squares method, Bayesian estimation, such as BayesA or BayesB or modification thereof, or Best Linear Unbiased Prediction (BLUP), for example marker-assisted Best Linear Unbiased Prediction (MA-BLUP), preferably Bayesian estimation, such as BayesA or BayesB or modifications thereof. Moreover, the determined udder health, fertility, other health, estimated breeding value and/or the individual effect of each genetic marker allele on said breeding value is stored in a non-volatile memory such as a computer memory and/or a database.

In another aspect, the present invention relates to a method of determining a genomic estimated breeding value (GEBV) of a bovine subject based on the genotype of said bovine subject for a plurality of genetic markers, said method comprising

a. providing a bovine subject,
b. obtaining a sample from said bovine subject comprising genetic material,
c. determining on the basis of said genetic material the genotype of said bovine subject for said plurality of genetic markers,
d. determining said GEBV by correlating said genotype for said plurality of genetic markers with a predetermined effect of each individual genetic marker allele on udder health, fertility, other health and/or estimated breeding value of at least 100 reference bovine subjects, said effect being determined as defined in any one of the preceding claims.

In a preferred embodiment of the aspects of the present, udder health comprises resistance to clinical mastitis, for example, udder health is determined by an udder health index weighing together information from resistance to clinical mastitis in first, second and/or third parity, somatic cell count (SCC), dairy form, udder support/fore udder attachment, and/or udder depth. Moreover, fertility is in one embodiment determined by a fertility index comprising Number of inseminations cows (AISC), Number of inseminations heifers (AISH), Fertility treatment 1st lactation (FERT1), Fertility treatment 2nd lactation (FERT2), Fertility treatments 3rd lactation (FERT3), Heat strength cows (HSTC), Heat strength heifers (HSTH), Calving to first insemination (ICF), First to last insemination cows (IFLC), First to last insemination heifers (IFLH), 56 day Non-return rate cows (NRRC), and/or 56 day Non-return rate heifers (NRRH), wherein the specific traits are defined more closely herein below. Also, in one embodiment of the aspects of the present invention, the other health is determined by an other health index comprising reproductive diseases, digestive diseases, and/or feet and leg diseases.

The plurality of genetic markers or genetic marker alleles is selected from any combination of polymorphic genetic markers, but in a preferred embodiment is a plurality of single nucleotide polymorphisms (SNP), microsatellite markers and/or mixtures thereof, most preferred SNP markers.

The determination of a genomic estimated breeding value according to the present invention is based on any combination of markers, but in a preferred embodiment, the plurality of genetic markers comprise a dense set of genetic markers located across the entire genome, for example comprising on average at least 1 genetic marker per cM, such as at least 10 genetic markers per cM, for example between 1 and 100 genetic markers per cM. Thus, in one embodiment, the plurality of genetic markers or genetic marker alleles comprises at least 50, such as at least 100, such as at least 200, such as at least 300, such as at least 400, such as at least 500, such as at least 600, such as at least 700, such as at least 800, such as at least 900, such as at least 1000, such as at least 2000, for example at least 3000, such as at least 4000, for example at least 5000, such as at least 6000, for example at least 7000, such as at least 8000, for example at least 9000, such as at least 10000, for example at least 12000, such as at least 14000, for example at least 16000, such as at least 18000, for example at least 20000, for example at least 22000, such as at least 24000, for example at least 26000, such as at least 28000, for example at least 30000, for example at least 32000, such as at least 34000, for example at least 36000, such as at least 38000, for example at least 40000, for example at least 42000, such as at least 44000, for example at least 46000, such as at least 48000, for example at least 50000, for example at least 52000, such as at least 54000, for example at least 56000, such as at least 58000, for example at least 60000, for example at least 62000, such as at least 64000, for example at least 66000, such as at least 68000, for example at least 20000, for example at least 72000, such as at least 74000, for example at least 76000, such as at least 78000, for example at least 80000, for example at least 82000, such as at least 84000, for example at least 86000, such as at least 88000, for example at least 90000, for example at least 92000, such as at least 94000, for example at least 96000, such as at least 98000, for example at least 100000 genetic markers or genetic marker alleles, or the plurality of genetic markers or genetic marker alleles comprises between 10000 and 100000, such as between 20000 and 80000, for example between 30000 and 60000, for example between 30000 and 50.000, such as between 30000 and 40000, for example between 35000 and 40000, for example between 37000 and 39000 genetic markers or genetic marker alleles. Several methods of detecting multiple genetic markers are available to the skilled person. In a preferred embodiment, the genetic marker alleles are detected simultaneously by gene chip technology, for example by using the Bovine SNP50 BeadChip provided by Illumina Inc.

The bovine subjects of the present invention belong to any cattle breed or family. In a preferred embodiment, the bovine subject is a member of the Holstein breed, for example a member of the Danish Holstein cattle population. The sample obtained in the methods of the present invention is any sample comprising genetic material, which may be extracted from the sample and used for genotyping of the bovine subject for the genetic markers of the invention. In a preferred embodiment, the sample is selected from the group consisting of blood, semen (sperm), urine, liver tissue, milk, muscle, skin, hair, follicles, ear, tail, fat, testicular tissue, lung tissue, saliva, spinal cord biopsy, and any other tissue; in a preferred embodiment, the sample is blood and/or milk.

The udder health, fertility, other health and/or genomic estimated breeding value (GEBV) is preferably calculated by simultaneous inclusion of all genetic marker effects regardless of statistic significance, and/or wherein genetic marker effects are calculated simultaneously, for example, udder health, fertility, other health and/or genomic estimated breeding value is calculated using Least-squares, Bayesian estimation, such as BayesA or BayesB or modifications thereof, or a marker-assisted Best Linear Unbiased Prediction (MA-BLUP), preferably Bayesian estimation, such as BayesA or BayesB or modifications thereof.

In a specific embodiment, the genomic estimated breeding value (GEBV) is combined with an estimated breeding value determined on the basis of an observed phenotype of said bovine subject and/or its offspring or other relatives.

In a third aspect, the present invention relates to a method for selective breeding, comprising determining a genomic estimated breeding value (GEBV) of a bovine subject using a method of the present invention, using said bovine subject as sire or dam for breeding if the GEBV of said bovine subject is equal to, or differs less than a predetermined amount from, a desired breeding value for the offspring. The specific GEBV depends on the statistical methods employed for determining the phenotypes and breeding values, and is apparent for those of skill in the art. In one embodiment, the bovine subject is used as sire or dam before an udder health, fertility and/or other health phenotype associated with the GEBV becomes manifest, and/or wherein said bovine subject does not have any offspring.

In a fourth aspect, the present invention relates to a computer program product including program code portions for performing, when run on a programmable apparatus, a method of the invention.

In a fifth aspect, the present invention relates to a computer readable medium comprising data representing a computer program product of the present invention.

In a further aspect, the present invention relates to a computer system and/or a programmable apparatus for performing a method of the present invention, comprising a computer program product and/or computer readable medium of the present invention.

In yet another aspect, the present invention relates to a kit comprising means for detecting a plurality of genetic markers, said kit comprising a computer program and/or a computer readable medium of the present invention.

A further aspect of the present invention relates to use of use of a computer program product, a computer readable medium, computer system and/or a programmable apparatus, and/or kit of the present invention for estimating a breeding value for a specific phenotype of a bovine subject.

The invention also relates to methods of determining a phenotypic trait, based on detection of one or more genetic markers, and methods for selected a bovine subject for breeding, as well as diagnostic kits for performing those methods.

Thus, another aspect of the present invention relates to a method of determining a phenotypic trait in a bovine subject, comprising detecting in a sample from said bovine subject the presence or absence of at least one genetic marker allele or a specific combination of genetic marker alleles, wherein said genetic marker allele or a specific combination of genetic marker alleles is associated with said phenotypic trait of said bovine subject and/or offspring or other relatives therefrom.

In another aspect, the present invention relates to a method for selecting bovine subjects for breeding purposes, said method comprising detecting in a sample from said bovine subject the presence or absence of at least one genetic marker allele or a specific combination of genetic marker alleles as defined in any of the preceding claims, wherein said at least one genetic marker allele or a specific combination of genetic marker alleles is associated with at least one trait of said bovine subject and/or offspring or other relatives therefrom.

A further aspect of the present invention relates to a diagnostic kit for determining the presence or absence in a bovine subject of at least one genetic marker allele or a specific combination of genetic marker alleles, wherein said genetic marker allele or a specific combination of genetic marker alleles is associated with a phenotypic trait of said bovine subject and/or offspring or other relatives therefrom.

### DESCRIPTION OF DRAWINGS

FIG. 1. Relationship between the DNA-information from older sires with confirmed breeding values and younger sires without breeding records.

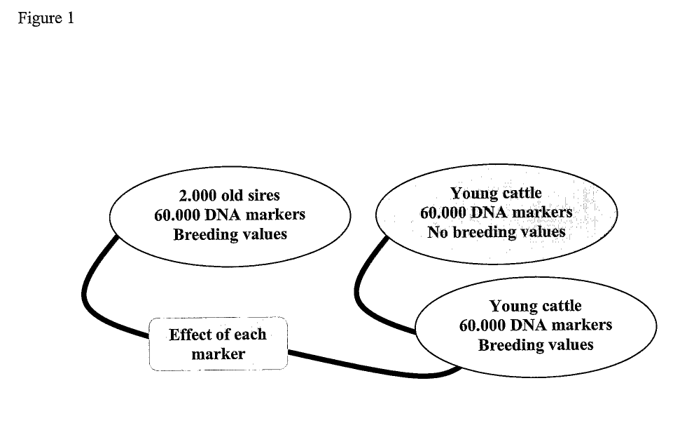

FIG. 2. Distributions of SNP effects for fertility estimated from models with different priors for SNP variance. Y-axis: frequency, X-axis: absolute SNP effect in standard deviation unit.

### DETAILED DESCRIPTION OF THE INVENTION

The present invention relates to genetic determinants of phenotypes and/or phenotypic traits in dairy cattle. The phenotypic traits of the present invention are predominantly economically important factors in the dairy industry. Furthermore, bovine subjects with genetic predisposition for non-desired traits, such as low production traits, are carriers of genetic determinants of such non-desired traits, which can be passed on to their offspring. Therefore, it is of economic interest to identity those bovine subjects that have a genetic predisposition for specific desirable quantitative traits, to aid in the selection of cattle with a genetic profile, which positively affects specific phenotypic traits. The invention specifically relates to a method of determining a phenotypic trait in a bovine subject, comprising determining a multiplicity of genetic marker alleles that are associated with a specific phenotypic trait of said bovine subject and/or offspring or other relatives therefrom. Preferably, the present invention relates to prediction of a genomic breeding value, wherein a combination or plurality of genetic markers located throughout the entire bovine genome is determined, and the effect of each individual genetic marker allele is included in the determination of breeding value. Thus, the present invention relates to a method for predicting a genomic estimated breeding value of a bovine subject based on the genotype of said bovine subject for a plurality of genetic markers.

The methods, products and uses of the present invention allows for more efficient selection of genetically superior bovine subjects for breeding, and for generation of cattle with economically important phenotypes, such as cattle less susceptible to disease such as clinical mastitis and/or other diseases, and/or cattle with higher fertility/reproductive rate.

In cattle, it is possible to simultaneously genotype a plurality of genetic markers, for example a kit for genotyping more than 50.000 SNP markers (SNP: single nucleotide polymorphism) is commercially available. This opens an opportunity for effective selection using dense markers through the whole genome, such that selection is based on a plurality of markers covering the entire genome, this method also referred to as genomic selection. Genomic selection is based on breeding values that are directly estimated from genome-wide dense marker panels. Therefore, genetic evaluation can be performed as soon as DNA is obtained, which allows accurate selection in both genders early in life. Genomic selection leads to considerably higher genetic gains than conventional quantitative genetic selection, and using genomic selection in dairy cattle breeding will considerably facilitate the genetic progress while reducing the cost for proving bulls.

BTA is short for Bos taurus autosome.

The term “heritability” is used herein to describe the strength with which traits are inherited and it varies depending on the trait in question. In general traits associated with reproduction and survival have low heritabilities, while milk production and early body size have medium heritabilities, and later growth and carcase traits (i.e. fat and muscle) have relatively high heritabilities.

The term “determining a genotype” as used herein, refers generally to the determination of which specific allele a subjects carries in a specific genomic polymorphic locus. In a locus comprising a polymorphic genetic marker, such as a single nucleotide polymorphism (SNP), homozygous bovine subjects are carriers of the same allele of the genetic marker on both chromosomes, while heterozygous bovine subjects are carriers of different genetic marker alleles. Genotyping of the bovine subject includes identifying the specific genetic marker allele that the subject is a carrier of.

The word “comprising” does not exclude the presence of other features or steps then those listed in a claim. The words ‘a’ and ‘an’ shall not be construed as limited to ‘only one’, but instead are used to mean ‘at least one’, and do not exclude a plurality. The mere fact that certain measures are recited in mutually different claims does not indicate that a combination of these measures cannot be used to advantage.

Estimated breeding value (EBV) and genomic estimated breeding value (GEBV). The appearance and performance of a bovine subject is influenced by multiple both genetic and environmental factors. The term “estimated breeding value” is also abbreviated EBV throughout herein. An estimated breeding value is an estimate of an animals (herein bovine subjects) genetic merit for a range of commercially relevant phenotypes or production traits. An estimated breeding value is used as a measure of the genetic potential of an animal, for example as a measure of its genetic capacity for calving, susceptibility to disease etc., which it can pass on to its offspring. Estimated Breeding Values (EBV's) and indexes are normally calculated from animals' individual performance records as well as those of their known relatives, where the environmental effects (feeding, management, disease, climate etc) are sifted out to leave an estimate of the genetic value for each trait.

EBV are normally calculated using information from several sources, such as measurements from the animal itself, measurements from the animal's herd mates (contemporaries), measurements from the animal's relatives and their contemporaries, the degree to which one trait influences another (correlation), and/or the degree to which each trait is passed on to the next generation (i.e. heritability). A conventional EBV calculation involves solving a set of simultaneous equations where the unknown variables are the genetic value of the animal and the environmental effect on its performance. When carried out many times, using all the information on the animal, the equations are able to quantify the unknown genetic component. Thus, an estimated breeding value is an estimation of how much better than the average an animal's genetics should be, based on the animal's performance, as well as the performance of all its relatives. The more closely related the relative is to the individual, the more it can contribute to the EBV. Therefore siblings, progeny and parents are normally used for calculating EBVs, as they share the most genes with the animal. The accuracy of the EBV increases with the number of relatives included in the phenotypic registration records. The end result of the calculations is an EBV and over time, as more pedigree and performance data is added, the solution to the equations becomes more accurate and the EBV approaches the true (empiric/observed) breeding value of the bovine subject.

Thus, the conventional methods for estimating a breeding value of a subject are based on the phenotypic record of the individual and the records of its relatives. For example, an animal has one record of protein yield 300 kg, its mother has one record 280 kg, and the animal does not have any other relatives' record in the data available. Assume that the population mean is 250 kg and the variance is Vp=900, and heritability of protein yield is h2=0.3. Breeding value of protein yield for this animal is estimated as

EBV=b1(300−250)+b2(280−250).

b1 and b2 are solved by mixed model equations

b1Vp+b20.5h2Vp=h2Vp

b10.5h2Vp+b2Vp=0.5h2Vp,

which leads to b1=0.284, b2=0.107, and EBV=0.284*50+0.107*30=17.41

In fact, an animal usually has a number of relatives in the dataset, EBV of all animals of interest can be estimated using an appropriate model, such as a BLUP model integrating a genetic relationship matrix which is constructed from the pedigree of the animals.

In conventional genetic prediction, breeding value of a candidate bull without progeny records can be estimated from parent average EBV (i.e., pedigree index). If EBV of sire and dam or maternal grandsire are available, conventional EBV of a candidate bull without progeny records are usually estimated as:

EBV=½×sire EBV+¼×maternal grandsire EBV

or

EBV=½×sire EBV+½×dam EBV

When EBV of a bovine subject is obtained from its sire and maternal grandsire EBV, the reliability of the EBV is equal to ¼ reliability of sire EBV+ 1/16 reliability of maternal grandsire EBV.

The present invention provides a method for predicting a genomic estimated breeding value for a bovine subject based on the genotype of said bovine subject for a plurality of genetic markers. An estimated breeding value (EBV) of the present invention, which is based on a plurality of genetic marker alleles is herein referred to as a genomic estimated breeding value (GEBV), because the EBV is based on genomic information, i.e. the EBV is calculated from the genotype of a bovine subject. Thus, the abbreviation, GEBV, as used herein, refers to a breeding value, which is estimated on the basis of genomic information, such as the genotype of a plurality of genetic markers. Methods of predicting a GEBV are provided herein below.

### Determination of Reference Effects

Prediction of a genomic estimated breeding value is based on a plurality of genetic markers, such as dense markers through the whole genome and marker effects. Marker effects are estimated from reference animals which have both phenotypic record and genotype record. For example, based on one or more reference animals with both phenotypic record and genotype records of 50000 markers, allele effects of each marker are estimated and shown in the following table.

### Estimated Effects of Alleles at Each Marker

| | M1 | M2 | M3 | . . . | M49998 | M49999 | M50000 |
|---|---|---|---|---|---|---|---|
| Allele 1 | −5 | 3 | 6 | . . . | −2 | 8 | −4 |
| Allele 2 | 5 | −3 | −6 | . . . | 2 | −8 | 4 |

An individual has marker types as presented in the following table

### Number of Allele 1 and Allele 2 at Each Marker for the Given Individual

| | M1 | M2 | M3 | . . . | M49998 | M49999 | M50000 |
|---|---|---|---|---|---|---|---|
| Allele 1 | 1 | 2 | 2 | . . . | 1 | 1 | 0 |
| Allele 2 | 1 | 0 | 0 | . . . | 1 | 1 | 2 |

The predicted breeding of this individual is the sum of allele effects over all markers, GEBV=1*(−5)+1*5+2*3+0*(−3)+2*6+0*(−6)+ . . . +1*(−2)+1*2+1*8+1*(−8)+0*(−4)+2*2

In this approach, breeding value of an individual can be predicted, no matter whether the individual and its relatives have records or not. Thus, in one aspect, the present invention relates to a method for determining the individual effect of a plurality of genetic marker alleles on udder health, fertility and/or other health of at least 100 reference bovine subjects and/or its relatives, said method comprising

a. providing at least 100 reference bovine subjects,
b. obtaining a sample from said one or more bovine subjects comprising genetic material,
c. determining on the basis of said genetic material the genotype of said one or more reference bovine subjects for said plurality of genetic markers,
d. determining the udder health, fertility and/or other health of said reference bovine subject, and
e. determining the individual effect of said plurality of genetic marker alleles on said udder health, fertility and/or other health of said reference bovine subject.

In a preferred embodiment, an estimated breeding value (EBV) is calculated on the basis of said udder health, fertility and/or other health. The effect of each individual genetic marker allele on udder health, fertility, and/or other health is for example determined by calculating a reference estimated breeding value (EBV) of said one or more reference bovine subjects, wherein said reference EBV is used as response variable for determining the effect of each individual genetic marker allele on udder health, fertility, other health and/or the EBV. Udder health, fertility and other health phenotypes are described more in more detail herein below. The phenotype of the bovine subject is preferably determined by registration of phenotypic traits of said bovine subject and/or off-spring/progeny and/or other relatives of said bovine subject. Registration of phenotypic traits is described elsewhere herein. In a preferred embodiment, the phenotype is udder health, fertility, and/or a health index (other health) comprising reproductive diseases, digestive diseases, feet and leg diseases, see herein below, however an udder health phenotype is particularly preferred, such as an udder health index comprising susceptibility to clinical mastitis as described herein below. The individual effect of a plurality of genetic marker alleles on udder health, fertility and/or other health phenotype or phenotypic trait is preferably evaluated by registration of phenotypic traits of at least 5, such as at least 10, for example at least 20, such as at least 30, for example at least 40 offspring or other relatives of said bovine subject.

The accuracy of the estimated individual effect of each marker of the plurality of markers increased with the number of reference bovine subjects, for which the phenotype, such as udder health, fertility and/or other health have been correlated with the genotype. Thus, in a preferred embodiment, the individual effect of the plurality of genetic marker alleles on udder health, fertility and/or other health, and/or a corresponding breeding value are determined for a plurality of reference bovine subjects, such as at least 10, at least 20, at least 50, at least 80, for example at least 100 reference bovine subjects, such as at least 1000 bovine subjects, for example at least 2000 reference bovine subjects, such as at least 3000 bovine subjects, for example at least 4000 reference bovine subjects, such as at least 5000 bovine subjects, for example at least 6000 reference bovine subjects, such as at least 7000 bovine subjects, for example at least 8000 reference bovine subjects, such as at least 9000 bovine subjects, for example at least 10000 reference bovine subjects, such as at least 20000 bovine subjects, for example at least 30000 reference bovine subjects, such as at least 40000 bovine subjects, for example at least 50000 reference bovine subjects.

In one embodiment, the individual effects of the plurality of genetic marker alleles on udder health, fertility and/or other health and/or the corresponding breeding value are determined for 1000 and 6000 bovine subjects, such as between 2000 and 5000 bovine subjects. In a preferred embodiment, the genetic marker effects are determined on the basis of between 1000 and 10000 bovine subjects, such as between 2000 and 6000 bovine subjects, for example between 2500 and 8000 bovine subjects, such as between 2000 and 7000 bovine subjects, such as between 3000 and 5000 bovine subjects, such as between 4000 and 4500 bovine subjects. The specific number of bovine subjects required for the most accurate determination of marker effects depends on the phenotype/phenotypic trait to correlate with genotype, and the heritability of said phenotype/phenotypic trait. Moreover, the number of reference animals may depend on whether the phenotype is observed on a bull or a cow, where for example fewer bulls are required for accurate estimation of genotype effects on udder health than for cows, because the bulls phenotype is an average of more daughter's and other relatives' registration.

Genetic marker effects may be determined by any method available to those of skill in the art. In a preferred embodiment, the effect of each individual genetic marker allele on a phenotype such as udder health, fertility and/or other health or the estimated breeding value is determined by calculating a reference breeding value of the reference bovine subjects, wherein said reference breeding value is used as response variable for determining the effect of each individual genetic marker allele on udder health, fertility and/or other health and/or the reference estimated breeding value. Any statistical method or model available to the skilled person may be employed. For example, the reference breeding value is determined using Least-squares method, Bayesian estimation, such as BayesA or BayesB or modification thereof, or Best Linear Unbiased Prediction (BLUP), for example marker-assisted Best Linear Unbiased Prediction (MA-BLUP), preferably Bayesian estimation, such as BayesA or BayesB or modifications thereof.

The phenotypic registrations, such as udder health, fertility and/or other health phenotypes, and/or a reference estimated breeding values are preferably stored in a database, for use in any method for predicting a breeding value of a bovine subject. In one embodiment, the reference breeding value and/or the individual effect of each genetic marker allele on said breeding value is stored in a non-volatile memory such as a computer memory and/or a database.

### Prediction of Genomic Estimated Breeding Value

The present invention provides a methodology for the determining an estimated breeding value based on the genotype of a bovine subject. Such a breeding value based on genomic information of the bovine subject is herein referred to as a genomic estimated breeding value or GEBV.

Thus, determination of a GEBV can be performed as soon as a sample comprising genetic material such as preferably DNA and/or RNA is obtained from the bovine subject, and a plurality of genetic markers have been genotyped. It is not required that the candidate bovine subject have phenotypic records, or progeny phenotypic records.

As shown in Table 1, reliabilities of GEBV are much higher than reliabilities of the EBV estimated from parent average EBV, thus providing that genomic prediction of young candidates based on GEBV is more accurate than conventional approach.

TABLE 1
Reliability of EBV from parent average and reliability of GEBV

Reliability of GEBV

| | | Based on | Based on |
|---|---|---|---|
| Trait | Reliability of PA | r²_{GEBV, EBV} | PEV* |
| Fertility | 0.216 | 0.412 | 0.566 |
| Other-health | 0.191 | 0.426 | 0.593 |
| Udder-health | 0.237 | 0.435 | 0.557 |

*PEV: prediction error variance estimated by model

Thus, the present invention provides an improved methodology for determining a genomic estimated breeding value for a bovine subject, in the absence of phenotypic records of said bovine subject or its progeny and/or relatives. In one aspect, the invention relates to a method of determining a genomic estimated breeding value (GEBV) of a bovine subject based on the genotype of said bovine subject for a plurality of genetic markers, said method comprising

a. providing a bovine subject,
b. obtaining a sample from said bovine subject comprising genetic material,
c. determining on the basis of said genetic material the genotype of said bovine subject for said plurality of genetic markers,
d. determining said GEBV by correlating said genotype for said plurality of genetic markers with a predetermined effect of each individual genetic marker allele on udder health, fertility, other health and/or estimated breeding value of at least 100 reference bovine subjects, said effect being determined as defined elsewhere herein. In a preferred embodiment, udder health comprises resistance to clinical mastitis, for example, udder health is determined by an udder health index weighing together information from resistance to clinical mastitis in first, second and/or third parity, somatic cell count (SCC), dairy form, udder support/fore udder attachment, and/or udder depth.

In a preferred embodiment, the genomic estimated breeding value is determined in respect of a phenotype such as udder health, such as susceptibility to subclinical and/or clinical mastitis. In a specific embodiment, udder health is determined by an udder health index weighing together information from resistance to clinical mastitis in first, second and/or third parity, somatic cell count (SCC), dairy form, udder support/fore udder attachment, and/or udder depth. In another embodiment, the phenotype is fertility, or an other health index as disclosed herein.

The plurality of genetic marker alleles is preferably a plurality of single nucleotide polymorphisms (SNP), microsatellite markers and/or mixtures thereof. However, any suitable genetic marker may be employed in the analysis.

### Statistical Model for Genomic Prediction

Several statistical models and algorithms are suitable for determining a genomic estimated breeding values based on plurality of genetic markers and/or dense markers of the present invention. For example Best Linear Unbiased Prediction (BLUP), BayesA and BayesB may all be used for analysis of marker effects, and for determining a genomic estimated breeding value. A linear BLUP approach assumes that effects of all genetic markers (e.g. SNPs and/or microsatellites) are normal distributed with same variance. BayesA and BayseB allow each marker to have its own variances of allele effects, and each variance is a sample of a scaled inverse Chi-square distribution. BayesB also models most SNP having zero effect, but a few having moderate to large effects. To simplify the computing algorithm in BayesA and BayesB (especially, the Metropolis-Hastings step in BayesB), alternative Bayesian approaches similar to BayesA and BayesB may be employed for prediction of a GEBV according to the present invention; such modifications of BayesA or BayesB being apparent to those of skill in the art. Some approaches model SNP effects as a product of a scaled effect and a scaling factor (which can be understood as standard deviation of allele effects in a marker). It may assume that the prior distribution of scaling factors is either a normal distribution or a mixture of two normal distributions.

In one example, a Bayesian method, which captures the features of BayesA and BayesB but simplifies the computing algorithm, is used to estimate marker effects for genomic prediction, in a model such as:

$$y = 1 μ + {\sum }_{i = 1}^{m} {X}_{i} {q}_{i} {v}_{i} + e ,$$

where y is the EBV determined by the observed phenotype (e.g. udder health, fertility and/or other health), μ is the intercept, m is the number of SNP markers, qᵢ is the vector of scaled SNP effects (scaled by standard deviation) of marker i with qᵢ˜N(0,I), vᵢ (vᵢ>0) is a scaling factor (standard deviation) for SNP effects of marker i, and e is the vector of residual. The effects of SNP types of marker i are the products of vᵢ and qᵢ.

Scaling factors vᵢ are in this approach assumed to have either a common prior distribution or a mixture prior distribution. A common prior distribution across the variances of chromosome segment effects, which leads to a slight or moderate differentiation between small and large effects of markers, is assumed to be a positive half-normal distribution,

vᵢ˜TN(0,σᵥ²), vᵢ>0

Mixture prior distribution, which lead to a strong differentiation between small and large effects of markers, assume that a proportion (p₀, typically large) of markers have a very small effect, and a proportion (p₁, typically small) of markers have a moderate or large effect. This is achieved by assuming that the prior distribution of vᵢ was sampled from either a positive half-normal distribution with a small variance (σᵥ₀²) or a positive half-normal distribution with large variance (σᵥ₁²)

vᵢ˜π₀TN(0,σᵥ₀²)+π₁TN(σᵥ₁²)

The GEBV for individual k is in one embodiment defined as the sum of predicted effects of SNP over all markers

$${GEBV}_{k} = \hat{μ} + {\sum }_{i = 1}^{m} {x}_{i ( k . )} {q}_{i} {v}_{i}$$

Preferably, the common prior model or the mixture prior models is used to estimate SNP effect for genomic prediction, most preferably the common prior model.

In general, in the methods of the present invention, the genomic estimated breeding value based on genotyping of a bovine subject is calculated by simultaneous inclusion of both significant and non-significant genetic marker effects, and/or the genetic marker effects are fitted in the model (calculated) simultaneously. Suitable statistical tools or models for determining that genomic estimated breeding value comprise Least-squares method, Bayesian estimation, such as BayesA or BayesB or modification thereof, or Best Linear Unbiased Prediction (BLUP), for example marker-assisted Best Linear Unbiased Prediction (MA-BLUP), preferably Bayesian estimation, such as BayesA or BayesB or modifications thereof.

### Combination GEBV and EBV Based on Progeny Records.

The present invention also relates to methods of predicting a breeding value, wherein a GEBV is combined with an EBV based on progeny records.

Thus, in one embodiment, the present invention relates to an index, which combines GEBV and parent average EBV and/or EBV based on other phenotypic registrations among progeny, offspring and/or other relatives. By combining the GEBV with an EBV determined on the basis of progeny records or parent average, the accuracy of the predicted breeding value in increased. The present invention incorporates any suitable method available for those of skill in the art for calculating an index of GEBV and a progeny/relative based EBV, or EBV based on parent average EBV. Several approaches are thus available for blending GEBV and conventional EBV.

In one specific embodiment, a simple index van be constructed as

I=b1GEBV+b2PA,

where I represents the index combining GEBV and PA EBV, and b1 and b2 are solved correlation analysis.

In another embodiment, a bivariate model is used to fit GEBV and progeny based EBV or PA EBV data, where GEBV and progeny based EBV (or PA) of both reference animal and candidates are used as data information, and relationship between animals are integrated into the analysis.

In a further embodiment, the index of GEBV and a progeny based EBV/PA EBV is predicted by a one-step approach, wherein the model is used to estimate GEBV and extra contribution from relatives.

The method of the present invention for estimating a breeding value based on genotyping of a plurality of genetic markers of a bovine subject, thus comprises combining the genomic estimated breeding value (GEBV) with a breeding value estimated on the basis of an observed phenotype of said bovine subject and/or its offspring or other relatives.

### Reliability

Importantly, genomic prediction, i.e. determination of a GEBV, according to the present invention, can be used to increase reliability in determination of breeding values. Reliability is a statistical measure, which describes the extent to which a test is dependable, stable, and consistent. For example, reliability may be calculated as: Reliability=1−Se²/Vg, where Se is posterior standard deviation of GEBV, Vg is genomic variance estimated from data, see examples herein below.

The present invention can be used to estimate breeding values with high reliability, and according to the present invention breeding values can be determined with reliabilities of at least 0.5, such as at least 0.6, for example at least 0.7, for example at least 0.8, such as at least 0.9, for example at least 0.99, such as 1. Thus, the present invention comprises genotyping of at least one genetic marker locus, such as multiplicity of genetic marker loci to obtain an estimated breeding value with a reliability according to the present invention of at least 0.5. In fact, the genotyping of multiple genetic marker loci and/or determination of a GEBV according to the present invention significantly increases the reliability of breeding values determined according to the present invention.

Genomic selection or selection of bovine subjects for breeding based on GEBV may for example be used for selection of cows and bulls for breeding purposes, as described below.

### Bovine Subject

The term “bovine subject” refers to cattle of any breed and is meant to include both cows and bulls, whether adult or newborn animals, and sires as well as dams. A bovine subject of the present invention comprises animals with phenotypic registrations as well as animal with non phenotypic record, including newborn calves. No particular age of the animals are denoted by this term. One example of a bovine subject is a member of the Holstein breed. In one embodiment, the bovine subject is a member of the Holstein-Friesian cattle population. In another embodiment, the bovine subject is a member of the Danish Holstein cattle population. In yet another embodiment, the bovine subject is a member of the Swedish Holstein cattle population. In another embodiment, the bovine subject is a member of the Holstein Swartbont cattle population. In another embodiment, the bovine subject is a member of the Deutsche Holstein Schwarzbunt cattle population. In another embodiment, the bovine subject is a member of the US Holstein cattle population. In one embodiment, the bovine subject is a member of the Red and White Holstein breed. In another embodiment, the bovine subject is a member of the Deutsche Holstein Schwarzbunt cattle population. In one embodiment, the bovine subject is a member of any family, which includes members of the Holstein breed. In one embodiment the bovine subject is a member of the Danish Red population. In another embodiment the bovine subject is a member of the Finnish Ayrshire population. In yet another embodiment the bovine subject is a member of the Swedish Red population. In another embodiment, the bovine subject is a member of the Swedish Red and White population. In yet another embodiment, the bovine subject is a member of the Nordic Red population.

In a preferred embodiment, the bovine subject of the present invention is a member of the Holstein breed, such as a member of the Holstein-Friesian cattle population. In a most preferred embodiment, the bovine subject of the present invention is a member of the Danish Holstein cattle population. However, in another embodiment, the bovine subject is a member of the Swedish Holstein cattle population. Moreover, it is understood that the methods and kits of the present invention are applicable to bovine subjects in general and thus also applies to any bovine subject, which is more or less related to the Danish Holstein cattle population.

In one embodiment of the present invention, the bovine subject is selected from the group consisting of Swedish Red and White, Danish Red, Finnish Ayrshire, Holstein-Friesian, Danish Holstein and Nordic Red. In another embodiment of the present invention, the bovine subject is selected from the group consisting of Finnish Ayrshire and Swedish Red cattle. In another embodiment of the present invention, the bovine subject is selected from the group consisting of Finnish Ayrshire and Swedish Red cattle.

In a preferred embodiment, the bovine subject is a member of the Danish Holstein cattle population. In another preferred embodiment, the bovine subject is a member of the Swedish Holstein cattle population.

In one embodiment, the bovine subject is selected from the group of breeds shown in table 1a

TABLE 1a
Breed names and breed codes assigned by ICAR (International
Committee for Animal Recording)

| | Breed | National Breed |
|---|---|---|
| Breed | Code | Names Annex |
| Abondance | AB | — |
| Tyrol Grey | AL | 2.2 |
| Angus | AN | 2.1 |
| Aubrac | AU | |
| Ayrshire | AY | 2.1 |
| Belgian Blue | BB | |
| Blonde d'Aquitaine | BD | |
| Beefmaster | BM | |
| Braford | BO | |
| Brahman | BR | |
| Brangus | BN | |
| Brown Swiss | BS | 2.1 |
| Chianina | CA | |
| Charolais | CH | |
| Dexter | DR | |
| Galloway | GA | 2.2 |
| Guernsey | GU | |
| Gelbvieh | GV | |
| Hereford, horned | HH | |
| Hereford, polled | HP | |
| Highland Cattle | HI | |
| Holstein | HO | 2.2 |
| Jersey | JE | |
| Limousin | LM | |
| Maine-Anjon | MA | |
| Murray-Grey | MG | |
| Montbéliard | MO | |
| Marchigiana | MR | |
| Normandy | NO** | |
| Piedmont | PI | 2.2 |
| Pinzgan | PZ | |
| European Red Dairy Breed | [RE]* | 2.1, 2.2 |
| Romagnola | RN | |
| Holstein, Red and White | RW*** | 2.2 |
| Salers | SL** | |
| Santa Gertrudis | SG | |
| South Devon | SD | |
| Shorthorn | [SH]* | 2.2 |
| Simmental | SM | 2.2 |
| Sahiwal | SW | |
| Tarentaise | TA | |
| Welsh Black | WB | |
| Buffalo (Bubalis bubalis) | BF | |

*new breed code
**change from earlier code because of existing code in France
***US proposal WW

In one embodiment, the bovine subject is a member of a breed selected from the group of breeds shown in table 1b

TABLE 1b
Breed names
National Breed Names

| English Name | National names | |
|---|---|---|
| Angus | Including | Aberdeen Angus |
| | | Canadian Angus |
| | | American Angus |
| | | German Angus |
| Ayrshire | Including | Ayrshire in |
| | | Australia |
| | | Canada |
| | | Colombia |
| | | Czech Republic |
| | | Finland |
| | | Kenya |
| | | New Zealand |
| | | Norway (NRF) |
| | | Russia |
| | | South Africa |
| | | Sweden (SRB) and SAB |
| | | UK |
| | | US |
| | | Zimbabwe |
| Belgian Blue | French: | Blanc-bleu Belge |
| | Flemish: | Witblauw Ras van Belgie |
| Brown Swiss | German: | Braunvïeh |
| | Italian: | Razza Bruna |
| | French: | Brime |
| | Spanish: | Bruna, Parda Alpina |
| | Serbo-Croatian: | Slovenacko belo |
| | Czech: | Hnedy Karpasky |
| | Romanian: | Shivitskaja |
| | Russian: | Bruna |
| | Bulgarian: | B'ljarska kafyava |
| European Red Dairy Breed | Including | Danish Red |
| | | Angeln |
| | | Swedish Red and White |
| | | Norwegian Red and White |
| | | Estonian Red |
| | | Latvian Brown |
| | | Lithuanian Red |
| | | Byelorus Red |
| | | Polish Red Lowland |

In one embodiment, the bovine subject is a member of a breed selected from the group of breeds shown in table 1c

TABLE 1c
Breed names
National Breed Names

| English Name | National names | |
|---|---|---|
| European Red Dairy Breed | | Ukranian Polish Red |
| (continued) | | (French Rouge Flamande?) |
| | | (Belgian Flamande Rouge?) |
| Galloway: | Including | Black and Dun |
| | | Galloway |
| | | Belted Galloway |
| | | Red Galloway |
| | | White Galloway |
| Holstein, Black and White: | Dutch: | Holstein Swarbont |
| | German: | Deutsche Holstein, schwarzbunt |
| | Danish: | Sortbroget Dansk Malkekvaeg |
| | British: | Holstein Friesian |
| | Swedish: | Svensk Låglands Boskaap |
| | French: | Prim Holstein |
| | Italian: | Holstein Frisona |
| | Spanish: | Holstein Frisona |
| Holstein, Red and White | Dutch: | Holstein, roodbunt |
| | German: | Holstein, rotbunt |
| | Danish: | Roedbroget Dansk Malkekvaeg |
| Piedmont | Italian: | Piemontese |
| Shorthorn | Including | Dairy Shorthorn |
| | | Beef Shorthorn |
| | | Polled Shorthorn |
| Simmental | Including dual purpose and beef use | |
| | German: | Flekvieh |
| | French: | Simmental Française |
| | Italian: | Razza Pezzata Rossa |
| | Czech: | Cesky strakatý |
| | Slovakian: | Slovensky strakaty |
| | Romanian: | Baltata româneasca |
| | Russian: | Simmentalskaja |
| Tyrol Grey | German: | Tiroler Grauvieh |
| | | Oberimtaler Grauvieh |
| | | Rätisches Grauvieh |
| | Italian: | Razza Grigia Alpina |

### Phenotypes and Phenotypic Traits

The methods and products (kits, computer systems etc.) of the present invention relates to the determination of a genomic estimated breeding value based on the genotype of a plurality of genetic markers, wherein the genotype is correlated with a predetermined effect of said genotype on a phenotype (e.g. udder health, fertility and/or other health) and/or the corresponding estimated breeding value. An marker effects and/or estimated breeding value may be determined for any phenotype or phenotypic trait.

The genetic marker allele, plurality of genetic marker alleles, and/or combination of genetic marker alleles of the present invention may be used to determine a number of phenotypic traits and/or the breeding value for a number of phenotypes or phenotypic traits of a bovine subject. A number of specific traits may be associated with a specific phenotype, and a phenotypic trait may also comprise a number of secondary or subordinate traits. Therefore, a phenotype may also be an overall index, which incorporates one or more of said phenotypic traits and/or subordinate/secondary traits. The phenotypes or phenotypic traits of the present invention may be grouped into reproduction traits, production traits, milk traits, meat traits, health traits and exterior traits. A list of secondary phenotypic traits associated with each of those traits is provided in the tables below.

| | | Trait Names [Abbreviation] |
|---|---|---|
| Trait Class | Trait Types | (custom name) |
| Reproduction | Fertility | Conception Rate [CONCRATE] |
| | | Calving Ease [CALEASE] |
| | | Calves Born Alive, % [BORNLIVE] |
| | | Pregnancy Rate [PREGRATE] |
| | | Twinning [TWIN] |
| | | Ovulation Rate [OVR] (N/A) |
| | | Nonreturn Rate [NONR] (N/A) |
| | | Still Birth [SB] (N/A) |
| | | Heat Intensity [HTINT] (N/A) |
| | General | Age At Puberty [PUBAGE] |
| | | Structurally Soundness (Legs, Feet, |
| | | Penis And Prepuce) [SOUND] |
| | | FSH At Castration [FSHC] (N/A) |
| | | Paired Testis Weight [PTW] (N/A) |
| | | Paired Testis Volume [PTV] (N/A) |
| | | Body Weight @ Castration [BWC] |
| | | (N/A) |
| | | Teat Placement [TPL] (N/A) |
| | | Teat Length [TLGTH] (N/A) |
| | | Quality Of Udder [UQUAL] (N/A) |
| | | Udder Depth [UDPTH] (N/A) |
| | | Foot Angle [FANG] (N/A) |
| | | Somatic Cell Count [SCC] (N/A) |
| | | Udder Cleft [UC] (N/A) |
| | | Body Form Composite Index [BFCI] |
| | | (N/A) |
| | | Udder Attachment [UA] (N/A) |
| | | Stature [STA] (N/A) |
| | | Strength [STR] (N/A) |
| | | Bone Quality [BQ] (N/A) |
| | | Dairy Form [DYF] (N/A) |
| | | Udder Height [UHT] (N/A) |
| | | Udder Composite Index [UCI] (N/A) |
| | | Udder Width [UWDT] (N/A) |
| | | Daughter Pregnancy Rate [DPR] (N/A) |
| | Semen Quality | |

| | | Trait Names [Abbreviation] |
|---|---|---|
| Trait Class | Trait Types | (custom name) |
| Production | Growth | Retail Product Yield [YEILD] |
| | | Pre-Weaning Average Daily Gain [PWADG] |
| | | Post-Weaning Average Daily Gain [POADG] |
| | | Mean Body Weight [BWM] |
| | | Lean To Fat Ratio [L2FRATIO] |
| | | Yearling Weight [W365] |
| | | Weaning Weight [WWT] |
| | | Birth Weight [BW] |
| | | Average Daily Gain [ADG] |
| | | Slaughter Weight [SWT] (N/A) |
| | | Temperament [TMP] (N/A) |
| | | Withers Height [WHT] (N/A) |
| | | Hip Height [HIPHT] (N/A) |
| | | Body Length [BL] (N/A) |
| | | Chest Width [CHWDT] (N/A) |
| | | Shoulder Width [SHOWDT] (N/A) |
| | | Chest Depth [CHDPT] (N/A) |
| | | Hip Width [HIPWDT] (N/A) |
| | | Lumbar Width [LUMWDT] (N/A) |
| | | Thurl Width [THUWDT] (N/A) |
| | | Pin Bone Width [PINWDT] (N/A) |
| | | Rump Length [RUMLGT] (N/A) |
| | | Cannon Circumference [CANCIR] (N/A) |
| | | Chest Girth [CHEGRT] (N/A) |
| | | Abdominal Width [ABDWDT] (N/A) |
| | | Abdominal Growth [ABDGRT] (N/A) |
| | | Body Depth [BD] (N/A) |
| | | Rump Angle [RANG] (N/A) |
| | | Rump Width [RUMWID] (N/A) |
| | | Heel Depth [Hdpth] (N/A) |
| | Lifetime | Veterinary Treatments [VT] |
| | Production | Length Of Productive Life [PL] |
| | | Persistency [Per] (N/A) |
| | Life History | PTA Type [PTAT] |
| | Traits | Behavior [BEH] (N/A) |

| | | Trait Names [Abbreviation] |
|---|---|---|
| Trait Class | Trait Types | (custom name) |
| Milk Traits | Milk Yield | Milk Yield [MY] |
| | | Milking Speed [MSPD] (N/A) |
| | | Dairy Capacity Composite Index [DCCI] |
| | | (N/A) |
| | Milk Protein | Protein Yield [PY] |
| | | Protein Percentage [PP] |
| | | Energy Yield [EY] (N/A) |
| | | Protein Content [PC] (N/A) |
| | Milk Fat | Fat Percentage [FP] |
| | | Fat Content [FC] |
| | | Fat Yield [FY] |

| | | Trait Names [Abbreviation] |
|---|---|---|
| Trait Class | Trait Types | (custom name) |
| Meat Traits | Production | Carcase Dressing [DRESSING] |
| | Meat Quality | % USDA Choice [PERCHOICE] |
| | | Tenderness Score [TENDER] |
| | | Ribeye Area [REA] |
| | | Marbling Score [MARBL] |
| | | Fat Thickness [FATTH] |
| | | Longissimus Muscle Area [LMA] (N/A) |
| | | Adjusted Fat [FATADJ] (N/A) |
| | | Ether Extractable Fat [EEF] (N/A) |
| | | Stearic Acid [STA] (N/A) |
| | | Oleic Acid [OLA] (N/A) |
| | | % Unsaturated Fatty Acids [PERUFA] (N/A) |
| | | Percent Unsaturated To Saturated Fatty |
| | | Acids [RUSFA] (N/A) |
| | | Fat Trim Yield [FATYD] (N/A) |
| | | Rib Fat [RIBF] (N/A) |
| | | Protein % [PP] (N/A) |
| | Carcase | Carcase Weight [CWT] |
| | Characteris | KPH Fat, CWT Ratio [KPHCWT] (N/A) |

| | | Trait Names [Abbreviation] |
|---|---|---|
| Trait Class | Trait Types | (custom name) |
| Health | Red Blood Cell Mass | |
| | Parasite Resistance | |
| | Parasite Load | |
| | Mastitis | Somatic Cell Score [SCS] |
| | | Somatic Cell Count [SCC] |
| | | Clinical Mastitis [CM] (N/A) |
| | Disease Resistance | Bovine Spngiform Encephalopathy |
| | | [BSE] (Mad-Cow Disease) |
| | | General Disease Resistance [GDR] |
| | | (N/A) |

| | | Trait Names [Abbreviation] |
|---|---|---|
| Trait Class | Trait Types | (custom name) |
| Exterior | Pigmentation | Degree Of Spotting [DS] |
| | Conformation | Degree Of Spotting [DS] |

The methods and products (kits, computer systems etc.) of the present invention relates to the determination of a genomic estimated breeding value based on the genotype of a plurality of genetic markers, wherein the genotype is correlated with a predetermined effect of said genotype on the phenotype or estimated breeding value. A breeding value may be determined for any phenotype or phenotypic trait.

Thus, in one embodiment a genetic marker allele, genotype of a plurality of genetic markers and/or combination of genetic marker alleles of the present invention may be used for determining in a bovine subject a phenotype, phenotypic trait and/or an estimated breeding value associated with reproduction/fertility, wherein said phenotypic trait is selected from any of the phenotypic traits listed above. I.e. the phenotypic trait is in one embodiment selected from the group consisting of Conception Rate [CONCRATE], Calving Ease [CALEASE], Calves Born Alive, % [BORNLIVE], Pregnancy Rate [PREGRATE], Twinning [TWIN], Ovulation Rate [OVR] (N/A), Nonreturn Rate [NONR] (N/A), Still Birth [SB] (N/A), Heat Intensity [HTINT] (N/A), Age At Puberty [PUBAGE], Structurally Soundness (Legs, Feet, Penis And Prepuce) [SOUND], FSH At Castration [FSHC] (N/A), Paired Testis Weight [PTW] (N/A), Paired Testis Volume [PTV] (N/A), Body Weight @ Castration [BWC] (N/A), Teat Placement [TPL] (N/A), Teat Length [TLGTH] (N/A), Quality Of Udder [UQUAL] (N/A), Udder Depth [UDPTH] (N/A), Foot Angle [FANG] (N/A), Somatic Cell Count [SCC] (N/A), Udder Cleft [UC] (N/A), Body Form Composite Index [BFCI] (N/A), Udder Attachment [UA] (N/A), Stature [STA] (N/A), Strength [STR] (N/A), Bone Quality [BQ] (N/A), Dairy Form [DYF] (N/A), Udder Height [UHT] (N/A), Udder Composite Index [UCI] (N/A), Udder Width [UWDT] (N/A), and Daughter Pregnancy Rate [DPR] (N/A).

In another embodiment, the plurality of genetic marker alleles, genetic marker allele and/or combination of genetic marker alleles of the present invention may be used for determining in a bovine subject a phenotype, phenotypic trait and/or an estimated breeding value associated with production, wherein said phenotypic trait is selected from the group consisting of Retail Product Yield [YEILD], Pre-Weaning Average Daily Gain [PWADG], Post-Weaning Average Daily Gain [POADG], Mean Body Weight [BWM], Lean To Fat Ratio [L2FRATIO], Yearling Weight [W365], Weaning Weight [WWT], Birth Weight [BW], Average Daily Gain [ADG], Slaughter Weight [SWT] (N/A), Temperament [TMP] (N/A), Withers Height [WHT] (N/A), Hip Height [HIPHT] (N/A), Body Length [BL] (N/A), Chest Width [CHWDT] (N/A), Shoulder Width [SHOWDT] (N/A), Chest Depth [CHDPT] (N/A), Hip Width [HIPWDT] (N/A), Lumbar Width [LUMWDT] (N/A), Thurl Width [THUWDT] (N/A), Pin Bone Width [PINWDT] (N/A), Rump Length [RUMLGT] (N/A), Cannon Circumference [CANCIR] (N/A), Chest Girth [CHEGRT] (N/A), Abdominal Width [ABDWDT] (N/A), Abdominal Growth [ABDGRT] (N/A), Body Depth [BD] (N/A), Rump Angle [RANG] (N/A), Rump Width [RUMWID] (N/A), Heel Depth [Hdpth] (N/A), Veterinary Treatments [VT], Length Of Productive Life [PL], Persistency [Per] (N/A), PTA Type [PTAT], and Behavior [BEH] (N/A).

In another embodiment, the plurality of genetic marker alleles, genetic marker allele and/or combination of genetic marker alleles of the present invention may be used for determining in a bovine subject a phenotype, phenotypic trait and/or an estimated breeding value associated with milk, wherein said phenotypic trait is selected from the group consisting of Milk Yield [MY], Milking Speed [MSPD] (N/A), Dairy Capacity Composite Index [DCCI] (N/A), Protein Yield [PY], Protein Percentage [PP], Energy Yield [EY] (N/A), Protein Content [PC] (N/A), Fat Percentage [FP], Fat Content [FC], and Fat Yield [FY].

In yet another embodiment, the plurality of genetic marker alleles, genetic marker allele and/or combination of genetic marker alleles of the present invention may be used for determining in a bovine subject a phenotype, phenotypic trait and/or an estimated breeding value associated with meat quality, wherein said phenotypic trait is consisting of Carcase Dressing [DRESSING], % USDA Choice [PERCHOICE], Tenderness Score [TENDER], Ribeye Area [REA], Marbling Score [MARBL], Fat Thickness [FATTH], Longissimus Muscle Area [LMA] (N/A), Adjusted Fat [FATADJ] (N/A), Ether Extractable Fat [EEF] (N/A), Stearic Acid [STA] (N/A), Oleic Acid [OLA] (N/A), % Unsaturated Fatty Acids [PERUFA] (N/A), Percent Unsaturated To Saturated Fatty Acids [RUSFA] (N/A), Fat Trim Yield [FATYD] (N/A), Rib Fat [RIBF] (N/A), Protein % [PP] (N/A), Carcase Weight [CWT], and KPH Fat/CWT Ratio [KPHCWT] (N/A).

In another embodiment, the plurality of genetic marker alleles, genetic marker allele and/or combination of genetic marker alleles of the present invention may be used for determining in a bovine subject a phenotype, phenotypic trait and/or an estimated breeding value associated with health/fitness/udder health, wherein said phenotypic trait is selected from the group consisting of Somatic Cell Score [SCS], Somatic Cell Count [SCC] and Clinical Mastitis [CM].

In another embodiment, the plurality of genetic marker alleles, genetic marker allele and/or combination of genetic marker alleles of the present invention may be used for determining in a bovine subject a phenotype, phenotypic trait and/or an estimated breeding value associated with exterior traits, such as Pigmentation and conformation, wherein said phenotypic trait for example is Degree Of Spotting [DS].

The plurality of genetic marker alleles, genetic marker allele and/or combination of genetic marker alleles of the present invention may be used for determining in a bovine subject a phenotype, phenotypic trait and/or an estimated breeding value of a phenotypic trait, such as a yield trait, a fitness trait, and/or a conformation trait. The phenotypic trait is in one example selected from the group consisting of Birth ease, Body score, Calving ease, Fat, Fat percent, Fertility, Health, Leg, Longevity, Milk, Milk organ, Milk speed, Protein, Prot. percent, Temperament, Udder health, Yield, Average, and Other diseases.

In a preferred embodiment, the plurality of genetic marker alleles, genetic marker allele and/or combination of genetic marker alleles of the present invention may be used for determining in a bovine subject a phenotype, phenotypic trait and/or an estimated breeding value of a phenotypic trait associated with mastitis, fertility/reproduction, calving and/or other diseases.

Genomic prediction of a breeding value according to the present invention is particularly suitable for determination of genetic determinants or estimation of a breeding value for complex genetic traits and phenotypes, and for traits for which the genetic correlations are relatively weak, such as for example fertility and mastitis.

### Fertility Phenotypic Traits

Fertility or the fertility phenotypes in a bovine subject is affected by a number of traits. The terms “fertility trait” or “phenotypic trait associated with fertility” or “fertility phenotype” as used herein refers to any trait or phenotype, which affect fertility in a bovine subject or its offspring or other relatives. In particular, fertility of a bovine subject in the context of the present application may be physically manifested by the fertility of its offspring or other relatives—both female and male. Thus, the fertility of a bull may be measured by a specific fertility trait in its female offspring or other relatives and/or the female offspring or other relatives of its offspring or other relatives. The calving traits may, thus, be assessed both as a “direct’ effect (D) of the sire in the calf and as a “maternal” effect (M) of the sire in the mother of the calf.

The breeding value of the present invention is determined for any trait, which affects fertility and/or is associated with fertility. In one embodiment, the breeding value is determined for an index of fertility, which incorporates one or more of the fertility traits described herein below. Specifically, the present invention and/or breeding values determined according to the present invention relates to traits such as those listed below:

Number of inseminations cows (AISC)
Number of inseminations heifers (AISH)
Fertility treatment 1st lactation (FERT1)
Fertility treatment 2nd lactation (FERT2)
Fertility treatments 3rd lactation (FERT3)
Heat strength cows (HSTC)
Heat strength heifers (HSTH)
Calving to first insemination (ICF)
First to last insemination cows (IFLC)
First to last insemination heifers (IFLH)
56 day Non-return rate cows (NRRC)
56 day Non-return rate heifers (NRRH)

AISc, ICF, IFLc, NRRc and HSTc in different parities are taken as the same traits. Fertility treatments in different parity are treated as different traits.

The individual fertility trait is described in more detail below.

### Number of Inseminations (AIS):

This trait is based on how many inseminations a cow or heifer needs in order to get pregnant; it describes the cows or heifer's ability to get pregnant after a number of inseminations (defined as pregnancy rate) and it also describes heat strength. In order to inseminate a cow at the correct time point it must show heat, and that is why this trait also reflects heat strength.

Cows(C) and heifers (H) are considered separately as AISC or AISH.

All inseminations are recorded by an artificial insemination technician (AI-technician) or a licensed farmer. This is later recorded in the national recording database and in this case recalculated into a breeding value for every sire.

Fertility Treatments 1^{st}, 2^{nd} and 3^{rd} Lactation (FERT1, FERT2, FERT3)

Fertility treatments are divided into three groups. Group 1 represents hormonal reproductive disorders and consists of ovarian cysts treatments. Group 2 represents infective reproductive disorders and consists of recordings of endometritis, metritis and vaginitis treatments. The last group consists of treatments for abortion, uterine prolaps, uterine torsion and other reproductive disorders. A disorder code is 1 if the cow has the corresponding disease or otherwise 0. The three lactations are considered as different traits. The trait may be recalculated into a breeding value for every sire.

In Denmark fertility treatments are recorded by veterinarians and AI-technicians. Thus the traits FERT1, FERT2 and FERT3 describe fertility treatments for first to third lactation, respectively.

### Heat Strength (HST)

HST measures the ability to show oestrus. The trait is measured subjectively by the individual farmer on a predefined relative scale from 1 to 5.

Cows and heifers are considered separately as HSTC or HSTH.

This may be recalculated into a breeding value for every sire.

### Calving to First Insemination (ICF)

This trait is only described for cows and reflects heat strength and reflects the ability to return to cycling after calving. In order to inseminate a cow it must return to cycling after calving. The recording unit is days. ICF may be recalculated into a breeding value for every sire.

### First to Last Insemination (IFL)

This is measured as the time from first insemination to the last insemination. The recording unit is days. IFL describes pregnancy rate and heat strength defined above. Cows and heifers are considered separately as IFLC or IFLH.

This is later recorded in the national recording database and in this case recalculated into a breeding value for every sire.

### 56-Day Non Return Rate (NRR)

NRR is based on whether the cow or heifer had a second insemination within 56 days after the first insemination. All cows and heifers not offered AI within 56 days were considered pregnant. NRR describes the cows' or heifers' ability to become pregnant after insemination, defined as pregnancy rate. The recording unit is days.

It is recorded by an artificial insemination technician (AI-technician) or a licensed farmer.

Cows and heifers are considered separately as NRRC or NRRH, respectively.

This is later recorded in the national recording database and in this case recalculated into a breeding value for every sire.

The genetic markers according to the present invention and/or fertility or breeding value determined according to the present invention are associated with at least one trait associated with fertility. In one embodiment, the trait associated with fertility is selected from the group consisting of Number of inseminations cows (AISC), Number of inseminations heifers (AISH), Fertility treatment 1st lactation (FERT1), Fertility treatment 2nd lactation (FERT2), Fertility treatments 3rd lactation (FERT3), Heat strength cows (HSTC), Heat strength heifers (HSTH), Calving to first insemination (ICF), First to last insemination cows (IFLC), First to last insemination heifers (IFLH), 56 day Non-return rate cows (NRRC) and 56 day Non-return rate heifers (NRRH)

In a specific embodiment, fertility is determined by the presence of a trait associated with fertility is Number of inseminations cows (AISC). In another specific embodiment, the trait associated with fertility is Number of inseminations heifers (AISH). In yet another specific embodiment, the trait associated with fertility is Fertility treatment 1st lactation (FERT1). In a further specific embodiment, the trait associated with fertility is Fertility treatment 2nd lactation (FERT2). In another specific embodiment, the trait associated with fertility is Fertility treatments 3rd lactation (FERT3). In another specific embodiment, the trait associated with fertility is Heat strength cows (HSTC). In yet another specific embodiment, the trait associated with fertility is Heat strength heifers (HSTH). In another specific embodiment, the trait associated with fertility is Calving to first insemination (ICF). In another specific embodiment, the trait associated with fertility is First to last insemination cows (IFLC). In another specific embodiment, the trait associated with fertility is First to last insemination heifers (IFLH). In another specific embodiment, the trait associated with fertility is 56 day Non-return rate cows (NRRC) and 56 day Non-return rate heifers (NRRH)

The fertility of a bovine subject as determined by the presence or absence of a genetic marker or genetic marker allele as defined by the present invention is estimated relative to the fertility of a bovine subject, wherein said genetic marker is absent from or present in the same locus, respectively. Thus, a bovine subject, wherein the presence of a genetic marker allele is associated with a reduced fertility, the reduction is estimated relative to a bovine subject, wherein said genetic marker is absent from the same genetic locus. Conversely, a bovine subject, wherein the absence of a genetic marker is associated with a reduced fertility, the reduction is estimated relative to a bovine subject, wherein said genetic marker is present from the same genetic locus.

### Measurement and Data Collection

The data of sub-traits of female fertility are preferably collected by A.I. technicians. Fertility treatments are carried out by veterinarians and/or by AI technicians. The data are preferably transferred to a Central Cattle Database, such as a computer readable medium or other physical or non-physical entities as described elsewhere herein.

The heat strength is in one embodiment recorded in Sweden. The judgement is preferably done by the farmer and is based on how clearly the cow shows oestrus signs. The heat strength is recorded as ordinal category codes. The data are reported to the milk recording and AI schemes in Sweden.

### Calculation of EBV and Fertility Index

In a preferred embodiment, fertility is determined by a fertility index comprising one or more fertility phenotypic traits selected from the group consisting of

In one embodiment, the sub-traits are divided into 3 groups, and breeding values of each trait were estimated using a multi-trait BLUP sire model for each group of traits, respectively. Group 1: NRRh, IFLh, NRRc, ICF, and IFLc. Group 2: AISh, HSTh, AISc, HSTc, ICF. Group 3 (FTR1, FTR2, and FTR3).

Model for group1 and group 2 is:

Y=Herd_Year+Month of first insemination+Parity_Age at first insemination+Proportion of breed+Proportion of heterozygosity+Sire+Residual,

where Herd_Year, Month of first insemination and Parity_Age at first insemination are fixed effect, Proportion of breed and Proportion of heterozygosity are fixed regression, Sire and Residual are random effects. For ICF, Month is the month of calving.

Model for group 3 is:

Y=Herd_{—}5Year+Month of calving+Parity_Age at calving+Proportion of breed+Proportion of heterozygosity+Sire+Residual,

where Herd_{—}5 Year, Month of calving and Parity_Age at calving are fixed effect, Proportion of breed and Proportion of heterozygosity are fixed regression, Sire and Residual are random effects.

In a preferred embodiment, a fertility index of the present invention is calculated as the weighted sum of sub-trait EBV, weighted by their economical weight.

### Mastitis or Udder Health Phenotype and Phenotypic Traits

Mastitis influences the udder health status of a bovine subject, and is affected by a number of traits. Traits that are associated with mastitis or udder health according to the present invention are for example the occurrence of clinical mastitis, somatic cell counts (SCC), somatic cell count, and udder conformation. Herein the term SCC is identical to the term CELL. Somatic cell score (SCS) is defined as the mean of log 10 transformed somatic cell count values (in 10,000/mL) obtained from the milk recording scheme. The mean was taken over the period 10 to 180 after calving. Traits associated with mastitis or udder health according to the present invention include traits, which affect udder health in the bovine subject or its offspring or other relatives. Thus, udder health and associated traits, such as mastitis traits of a bull are physically manifested by its female offspring or other female relatives.

In one embodiment of the present invention, udder health is reflected by the quantitative traits Mas1, Mas2 (CM1), Mas3 (CM2), Mas4 (CM3), SCC, SCS and/or udder health index. The quantitative traits are for example defined by the following parameters:

Mas1: Treated cases of clinical mastitis in the period −5 to 50 days after 1^{st} calving.
Mas2 (also designated CM1): Treated cases of clinical mastitis in the period −5 to 305 days after 1^{st} calving.
Mas3 (also designated CM2): Treated cases of clinical mastitis in the period −5 to 305 days after 2^{nd} calving.
Mas4 (also designated CM3): Treated cases of clinical mastitis in the period −5 to 305 days after 3^{rd} or later calving.
SCS: Mean SCS in period 5-180 days after 1^{st} calving.

In a preferred embodiment, the genomic estimated breeding value (GEBV) according to the present invention is determined for udder health, wherein udder health is determined by an udder health index. In a preferred embodiment, the udder health index includes the following 4 sub-traits:

1) Mastitis during the period from 10 days before calving to 50 days after calving in first parity.
2) Mastitis during the period from 10 days before calving to 305 days after calving in first parity.
3) Mastitis during the period from 10 days before calving to 100 days after calving in second parity.
4) Mastitis during the period from 10 days before calving to 100 days after calving in third parity.

In addition, further parameters may be included in the udder health index to improve the accuracy or reliability of the udder health assessment or GEBV, for example the following 4 type traits may be used as information-traits (secondary traits) to improve estimated breeding value (EBV) of the udder health index based on the mastitis-traits mentioned above:

1) Somatic cell count during the period from 10 days to 180 days after calving in first parity.
2) Dairy form measured in first parity.
3) Udder support (fore udder attachment) measured in first parity.
4) Udder depth measured in first parity.

Thus, in one embodiment, the udder health index of the present invention is an index weighing together information from Mas1-Mas4, SCC, fore udder attachment, udder depth, and udder band, as defined herein.

In a preferred embodiment, the individual effect of a plurality of genetic marker alleles on udder health and/or estimated breeding value, and/or the estimation of a genomic breeding value by correlation of a genotype with a predetermined effect of said phenotype is determined with respect to udder health, such as an udder health index. The udder health index preferably comprises clinical mastitis, such as susceptibility to clinical mastitis, or resistance to clinical mastitis. Moreover, the EBV or GEBV of udder health determined according to the present invention is in a preferred embodiment calculated as an index including the four mastitis-traits, e.g. the weighted sum of EBV or GEBV of the four mastitis-traits, weighted by their economical importance.

### Measurement and Data Collection

Clinical mastitis is preferably diagnosed by veterinarians. The data is preferably binary data, which define presence or absence of clinical mastitis. Registrations are for example transferred from the veterinarians' computer to a Central Cattle Database, and/or a computer readable medium or memory.

Analysis of somatic cell count is carried out at central laboratories. The information is preferably automatically transferred to the Central Cattle Database. In the prediction of breeding value, test-day somatic cell counts are preferably generalized into a geometric mean.

For the type traits (e.g., Dairy form, Udder support, and Udder depth), cows in first lactation are preferably classified according to a linear scale (ordinal categorical scores) by the classifiers of the individual breed. The scores of a daughter group classification are preferably entered into the Central Cattle Database and/or a computer readable medium or memory by means of a portable terminal.

### Calculation of EBV and Udder-Health Index:

Estimated breeding value (EBV) or GEBV of the sub-traits are for example estimated using a multi-trait best linear unbiased prediction (BLUP) sire model including the 4 mastitis trait and the 4 information-traits. The model is:

Y=Herd_Year_Season+Year_Month+Calving age (only first parity)+Proportion of breed+Proportion of heterozygosity+Sire+Residual,

where Herd_Year_Season, Year_Month and Calving age are fixed effect, Proportion of breed and Proportion of heterozygosity are fixed regression, Sire and Residual are random effects.

EBV or GEBV of Udder health is in a preferred embodiment calculated as an index including the four mastitis-traits, i.e., the weighted sum of EBV or GEBV of the four mastitis-traits, weighted by their economical importance.

In one embodiment of the present invention, a method, product, kit and/or breeding value described herein relates to udder health index. In another embodiment of the present invention, the method, product, kit and/or breeding value described herein relates to clinical mastitis. In another embodiment, the method, product, kit and/or breeding value of the present invention pertains to sub-clinical mastitis, such as detected by somatic cell score. In yet another embodiment, the method, product, kit and/or breeding value of the present invention primarily relates to clinical mastitis in combination with sub-clinical mastitis such as detected by somatic cell counts.

Registrations from daughters of bulls may be examined and used in establishing a relation between the observable incidents of mastitis and potential genetic determinants of udder health in a bovine subject.

### Calving Phenotypic Traits

Calving in a bovine subject is affected by a number of traits. Traits that are associated with calving according to the present invention are for example the occurrence of stillbirth (SB), calving difficulty (CD) and the size of the calf at birth (CS). The traits are assessed by a direct effect (D) of the sire in the calf. However, the traits are also assessed as a maternal effect (M) of the sire in the mother of the calf. By the term calving characteristics is meant traits which affect calving in the bovine subject or its offspring or other relatives. Thus, calving traits of a bull are physically manifested by its offspring or other relatives—both female and male. In the present invention calving characteristics comprise the traits SB, CD, and CS, which refer to the following characteristics:

SB: Designates stillbirths.
CS: Size of calves.
CD: Calving difficulties, which are based on registrations from the farmers where it is subjectively registered how difficult the calving is. The calving difficulties consist of four categories:
1: easy with no help
2: easy with assistance
3: difficult but without veterinary assistance
4: difficult with veterinary assistance

In one embodiment of the present invention, the method, genetic marker allele and/or combination of genetic marker alleles and kit described herein relates to still births, calving difficulties as categorized herein and/or calf size. In one embodiment of the present invention, the method, genetic marker allele and/or combination of genetic marker alleles and kit described herein relates to still births. In another embodiment, the method, genetic marker alleles and/or combinations of genetic marker alleles and kit of the present invention pertains to calving difficulties, such as detected by the calving difficulty categories described above. In yet another embodiment, the method, genetic marker alleles and/or combinations of genetic marker alleles and kit of the present invention relates to calf size. In another embodiment of the present invention, the method, genetic marker alleles and/or combinations of genetic marker alleles and kit described herein relates to any combination of still birth, calving difficulties and/or calf size.

### Other Health

In one preferred embodiment, the methods, kits, genetic marker alleles and/or combination of genetic marker alleles/haplotypes of the present invention relates to determining a phenotype or GEBV for an other health phenotype.

Other health phenotype is preferably determined by a “other health index”, which includes 9 sub-traits (3 types×3 parities). These sub-traits are: 1) reproductive diseases, 2) digestive diseases, 3) feet and leg diseases in the period 10 days before calving to 100 days after calving in first, second and third parity.

1) Reproductive diseases include abortion, endometritis, uterine prolapse, uterine torsion, endometritis treatment, follicular cysts, retained placenta, caesarian section, vaginitis and other reproductive diseases.
2) Digestive diseases include diarrhoea, traumatic reticuloperitonitis, ludigestion, hypomagnesemia, ketosis, milk fever, abomasal displacement, abomasal indigestion, rumen acidosis, enteritis, bloat and other digestive and metabolic diseases.
3) Feet and leg diseases include heel erosion, interdigital dermatitis, claw trimming by veterinarian, interdigital necrobacillosis, interdigital skin hyperplasia, laminitis, arthritis, sole ulcer, pressure injuries, tenosynovitis of hoofs and other leg diseases.

Mastitis in first parity is in one embodiment used as an information trait to improve the accuracy of the observed phenotype, EBV, and/or GEBV of the above disease-traits.

### Measurement and Data Collection

Digestive diseases and feet and leg diseases are preferably diagnosed by veterinarians. Reproduction disorders are preferably detected by veterinarians and by AI technicians. The data (binary data) are preferably transferred to a Central Cattle Database. In genetic evaluation, all codes for each type of diseases within lactation are pooled. If the sum is larger or equal to 1, the cow is considered sick (=1). Otherwise she is considered healthy (=0).

### Calculation of EBV and Other Health Index

Breeding value for each sub-trait is in one embodiment predicted using a multi-trait BLUP sire model including the 9 sub-traits and mastitis in first parity. For example, the model is:

Y=Herd_Year_Season+Year_Month+Calving age (only first parity)+Proportion of breed+Proportion of heterozygosity+Sire+Residual,

where Herd_Year_Season, Year_Month and Calving age are fixed effect, Proportion of breed and Proportion of heterozygosity are fixed regression, Sire and Residual are random effects. Index of other-health is calculated as the weighted sum of EBV of the 9 sub-traits, weighted by their economical importance.

### Genetic Markers

The term “genetic marker” refers to a variable nucleotide sequence (polymorphism) of the DNA on the bovine chromosome. The variable nucleotide sequence can be identified by methods known to a person skilled in the art, as explained elsewhere herein, for example by using specific oligonucleotides in for example amplification methods and/or hybridization techniques and/or observation of a size difference. However, the variable nucleotide sequence may also be detected by sequencing or for example restriction fragment length polymorphism analysis. In a preferred embodiment, the genetic marker allele is detected by gene chip technology or microarrays. The variable nucleotide sequence may be represented by a deletion, an insertion, repeats, and/or a point mutation. Thus, a genetic marker locus comprises a variable number of polymorphic alleles. Thus, the term “genetic marker allele” as used herein, refers to a specific such allele, i.e. a specific nucleic acid sequence.

In one embodiment, the genetic marker of the present invention is a quantitative trait locus. One type of genetic marker is a microsatellite marker that is associated with a quantitative trait locus. Thus, in one embodiment, the genetic marker of the present invention is a microsatellite. Microsatellite markers are short sequences repeated after each other. In short sequences are for example one nucleotide, such as two nucleotides, for example three nucleotides, such as four nucleotides, for example five nucleotides, such as six nucleotides, for example seven nucleotides, such as eight nucleotides, for example nine nucleotides, such as ten nucleotides. However, changes sometimes occur and the number of repeats may increase or decrease. The specific definition and locus of the polymorphic microsatellite markers can be found in the USDA genetic map (Kappes et al. 1997; or by following the link to U.S. Meat Animal Research Center http://www.marc.usda.gov/). A microsatellite locus comprises a variable number of polymorphic alleles. Thus, the term “microsatellite marker allele” as used herein refers to a specific such allele. In one embodiment of the at least one genetic marker of the present invention is detected by identification of a microsatellite marker allele, which is genetically coupled to said genetic marker.

In a preferred embodiment, the genetic marker of the present invention is a single nucleotide polymorphism (SNP). An SNP is a variation in the genetic code at a specific point on the DNA, i.e. a genetic change that is caused by substitution of a single nucleotide (such as an A is changed to G). An SNP locus comprises at least two alleles, and an SNP locus comprising two, three, and four alleles are referred to as bi-, tri-, or tetra-allelic polymorphisms, respectively. The bovine genome comprise large amounts of SNPs, and SNP markers are therefore highly suitable for use in selection for desirable phenotypic traits, which are genetically linked to the SNPs.

In one embodiment of the present invention, the specific genetic marker alleles are associated with quantitative trait loci affecting a phenotypic trait as defined below, including traits affecting udder health, such as susceptibility to mastitis, fertility, calving, and other diseases, as defined herein.

The term “associated with” as used herein in regards to the genetic marker allele and/or combination of genetic marker alleles and phenotypic traits, is meant to comprise both direct and indirect genetic linkages. Thus, a genetic marker allele and/or combination of genetic marker alleles which are associated with a trait according to the present invention may be coupled to said trait by direct or indirect genetic linkages. Moreover, the term “trait associated with” as used herein in regards to a specific phenotype, relates to any phenotypic traits, which to any extent contribute to said phenotype. For example, the traits somatic cell count (SCC), somatic cell score (SCS), udder conformation (which comprises several quantitative measures, such as fore udder attachment, udder depth, udder texture etc.), and diagnostic variables (such as treated cases of clinical mastitis within a specific timeframe) contribute to the overall mastitis phenotype. Thus, the “traits associated with mastitis”, or “mastitis phenotypic traits” comprise SCC, SCS, udder conformation and diagnostic variables, including the subindexes of any of said phenotypic traits.

The term “genetically coupled” is used herein about two genomic loci, which tend to segregate together. Thus, an SNP marker allele, which is genetically coupled to another genetic marker allele associated with a specific phenotypic trait according to the present invention, is indicative of said genetic marker, and may consequently be detected in a sample as an alternative of detecting said genetic marker associated with said phenotypic traits, for example traits associated with mastitis or fertility.

It is furthermore appreciated that the nucleotide sequences of the genetic marker allele or combination of marker alleles of the present invention are genetically associated with phenotypic traits of the present invention in a bovine subject. Consequently, it is also understood that a number of genetic markers may be comprised in the nucleotide sequence of the DNA region(s) flanked by and including the genetic markers according to the method of the present invention.

The present invention relates to methods for determining a genomic estimated breeding value based on the genotype of a bovine subject for a plurality of genetic markers, such as dense markers located across the entire bovine genome. The plurality of genetic markers is in one preferred embodiment a set of dense markers located across the entire bovine genome, such as located with at least one marker for every 1 cM on average, for example at least one marker per 0.1 cM. Thus, in one embodiment, the plurality of genetic marker alleles of the present invention is a plurality of single nucleotide polymorphisms (SNP), microsatellite markers and/or mixtures thereof, preferably a plurality of SNPs.

In one embodiment, the plurality of genetic markers or genetic marker alleles comprises at least 50, such as at least 100, such as at least 200, such as at least 300, such as at least 400, such as at least 500, such as at least 600, such as at least 700, such as at least 800, such as at least 900, such as at least 1000, such as at least 2000, for example at least 3000, such as at least 4000, for example at least 5000, such as at least 6000, for example at least 7000, such as at least 8000, for example at least 9000, such as at least 10000, for example at least 12000, such as at least 14000, for example at least 16000, such as at least 18000, for example at least 20000, for example at least 22000, such as at least 24000, for example at least 26000, such as at least 28000, for example at least 30000, for example at least 32000, such as at least 34000, for example at least 36000, such as at least 38000, for example at least 40000, for example at least 42000, such as at least 44000, for example at least 46000, such as at least 48000, for example at least 50000, for example at least 52000, such as at least 54000, for example at least 56000, such as at least 58000, for example at least 60000, for example at least 62000, such as at least 64000, for example at least 66000, such as at least 68000, for example at least 20000, for example at least 72000, such as at least 74000, for example at least 76000, such as at least 78000, for example at least 80000, for example at least 82000, such as at least 84000, for example at least 86000, such as at least 88000, for example at least 90000, for example at least 92000, such as at least 94000, for example at least 96000, such as at least 98000, for example at least 100000. In another embodiment, the plurality of comprises between 10000 and 100000, such as between 20000 and 80000, for example between 30000 and 60000, for example between 30000 and 50.000, such as between 30000 and 40000, for example between 35000 and 40000, for example between 37000 and 39000. In a specific embodiment, the plurality of comprises between 38000 and 38900 genetic markers or genetic marker alleles.

The SNP markers, which are genotyped in the present invention, are selected from any known genetic markers, including genetic markers available on commercially available detection systems, such as commercially available gene chips. In a preferred embodiment, the plurality of genetic markers is selected from the SNP markers of the Illumina Bovine SNP50 BeadChip (see e.g. http://illumina.com/downloads/BovineSNP5O_data_sheet.pdf). The BovineSNP50 BeadChip features more than 54,000 evenly spaced SNP probes that span the bovine genome. The BovineSNP50 BeadChip covers common SNPs validated in economically important beef and dairy cattle breed types and presents an average minor allele frequency (MAF) of 0.25 across all loci. Importantly, this BeadChip offers uniform coverage with an average probe spacing of 51.5 kb to provide more than sufficient SNP density for robust genome-association studies in cattle. The SNPs have been described by the Bovine HapMap Consortium, which, to date, has conducted extensive genotyping of cattle. Consequently, in a preferred embodiment, the genetic markers of the present invention are genotyped, i.e. the genetic marker alleles of the methods and products of the present invention are determined, by gene chip technology/DNA microarrays.

One aspect of the present invention relates to a method of determining a phenotypic trait as defined herein in a bovine subject, comprising detecting in a sample from said bovine subject the presence or absence of at least one genetic marker allele, such as a plurality of genetic marker alleles or a specific combination of genetic marker alleles, wherein said genetic marker allele, plurality or specific combination of genetic marker alleles is associated with said phenotypic trait of said bovine subject and/or offspring or other relatives therefrom. The present invention relates to any type of genetic marker. Preferred genetic marker alleles are however single nucleotide polymorphisms (SNP markers) and/or microsatellite markers.

In a preferred embodiment, the present invention relates to a specific combination of genetic markers. In this embodiment, the contribution of each individual genetic marker allele to a phenotypic trait as defined herein is used for the determination of a phenotypic trait and/or breeding value of a bovine subject. Therefore, the present invention also relates to methods and kits, which comprises determining multiple genetic marker alleles, and/or a specific combination of genetic marker alleles in a sample from said bovine subject.

Thus, in one embodiment, the present invention relates to methods, products and kits for determining a phenotypic trait comprising determining a specific combination or a plurality of at least 2 genetic marker alleles, such as at least 3 genetic marker alleles, such as at least 4 genetic marker alleles, such as at least 5 genetic marker alleles, such as at least 10 genetic marker alleles, such as at least 20 genetic marker alleles, such as at least 30 genetic marker alleles, such as at least 40 genetic marker alleles, such as at least 50 genetic marker alleles, such as at least 60 genetic marker alleles, such as at least 70 genetic marker alleles, such as at least 80 genetic marker alleles, such as at least 90 genetic marker alleles, such as at least 100 genetic marker alleles, such as at least 200 genetic marker alleles, such as at least 300 genetic marker alleles, such as at least 400 genetic marker alleles, such as at least 500 genetic marker alleles, such as at least 600 genetic marker alleles, such as at least 700 genetic marker alleles, such as at least 800 genetic marker alleles, such as at least 900 genetic marker alleles, such as at least 1000 genetic marker alleles, such as at least 2000 marker alleles, for example at least 3000 marker alleles, such as at least 4000 genetic marker alleles, for example at least 5000 genetic marker alleles, such as at least 6000 marker alleles, for example at least 7000 marker alleles, such as at least 8000 genetic marker alleles, for example at least 9000 genetic marker alleles, such as at least 10000 marker alleles, for example at least 12000 marker alleles, such as at least 14000 genetic marker alleles, for example at least 16000 genetic marker alleles, such as at least 18000 marker alleles, for example at least 20000 marker alleles, for example at least 22000 marker alleles, such as at least 24000 genetic marker alleles, for example at least 26000 genetic marker alleles, such as at least 28000 marker alleles, for example at least 30000 marker alleles, for example at least 32000 marker alleles, such as at least 34000 genetic marker alleles, for example at least 36000 genetic marker alleles, such as at least 38000 marker alleles, for example at least 40000 marker alleles, for example at least 42000 marker alleles, such as at least 44000 genetic marker alleles, for example at least 46000 genetic marker alleles, such as at least 48000 marker alleles, for example at least 50000 marker alleles, for example at least 52000 marker alleles, such as at least 54000 genetic marker alleles, for example at least 56000 genetic marker alleles, such as at least 58000 marker alleles, for example at least 60000 marker alleles, for example at least 62000 marker alleles, such as at least 64000 genetic marker alleles, for example at least 66000 genetic marker alleles, such as at least 68000 marker alleles, for example at least 20000 marker alleles, for example at least 72000 marker alleles, such as at least 74000 genetic marker alleles, for example at least 76000 genetic marker alleles, such as at least 78000 marker alleles, for example at least 80000 marker alleles, for example at least 82000 marker alleles, such as at least 84000 genetic marker alleles, for example at least 86000 genetic marker alleles, such as at least 88000 marker alleles, for example at least 90000 marker alleles, for example at least 92000 marker alleles, such as at least 94000 genetic marker alleles, for example at least 96000 genetic marker alleles, such as at least 98000 marker alleles, for example at least 100000 marker alleles. In one embodiment, the present invention comprises genotyping between 100 and 100000, such as between 10000 and 60000, for example between 20000 and 40000 genetic markers in one or more bovine subjects.

In one embodiment, the genetic markers of the present invention are selected from the group of genetic markers set out in the BovineSNP50 BeadChip from Illumina Inc. Each of the genetic markers set out in the BovineSNP50 BeadChip from Illumina Inc are also claimed as a single embodiment of the present invention.

It is understood that the genetic marker alleles of the present invention may be genetically coupled to other genetic polymorphisms, which may then serve as alternative genetic markers for determining a bovine subject with a specific phenotype according to the present invention. Such alternative genetic markers, however, cannot be used for selection without also selecting for the genetic marker alleles of the present invention, and therefore, said alternative genetic markers are also within the scope of the present invention.

### SNP Assay

In a preferred embodiment, the genetic markers or marker alleles for use in the present invention are determined by the BovineSNP50 BeadChip from Illumina Inc, said assay featuring more than 54,000 evenly spaced probes that target SNPs. The BovineSNP50 BeadChip presents an average SNP spacing of 51.5 Kb across the entire genome, thus allowing sufficient SNP density for genomic prediction of phenotypic traits of the present invention. The genetic markers of the BovineSNP50 BeadChip from Illumina Inc are available from the suppliers website.

The BovineSNP50 BeadChip targets evenly distributed SNPs that are polymorphic across the breeds tested and provides an average probe spacing of 51.5 kb and a median spacing of 37.3 kb. In general, observed linkage disequilibrium (LD) in multiple breeds of cattle suggests haplotype blocks of approximately 70 kb on average, indicating that the resolution offered by the BovineSNP50 chip is well within the resolution of LD in cattle.

### Selective Breeding

Selective breeding of cattle is based on selection of sires and dams with superior genetic backgrounds to pass on to their off-spring. Sires and/or dams are specifically selected on the basis of their genetic merit with respect to economically important phenotypes, such as resistance/susceptibility to disease (e.g. mastitis) and/or yield.

The present invention allows the selection of bovine subjects for breeding based on the genomic estimated breeding value (GEBV) of the sire and/or dam; i.e. the method of determining an genomic estimated breeding value for a bovine subject (e.g. without a known phenotype) of the invention, may be used in a method for selective breeding. According to an aspect of the invention, the method for selective breeding, here also referred to as breeding program, comprises selecting a sire and a dam, e.g. from a plurality of bovine subjects, wherein a bovine subject selected for breeding has a genomic estimated breeding value of a specific order of magnitude. The breeding values are chosen, in a manner known to the skilled person, such that offspring of the sire and dam may have a desired breeding value. After selection of the sire and dam on the basis of the determined genomic estimated breeding value, offspring is produced using the sire and dam. The breeding value of the offspring may be estimated as described hereinabove, e.g. before the phenotype, associated with the desired breeding value, becomes manifest in the offspring. The breeding program may proceed with the offspring as sire or dam for a next generation of offspring if its genomic estimated breeding value or estimated breeding value, or the GEBV or EBV of its offspring is larger than, equal to, or differs less than a predetermined amount from, the desired breeding value. The determination of an estimated breeding value before the phenotype is known allows for more efficient breeding of cattle, because an accurate breeding value can be determined on the basis of a genetic test. This, allows for more accurate selection of genetically superior sires and dams, and thus facilitate breeding of cattle with improved genetic potential in respect of economical and ethical important phenotypic factors, such as disease resistance, yield etc. Moreover, the time required for a breeding program is reduced since registration/observation of phenotypic traits is not required for determining the genomic estimated breeding value of an animal by a method of the present invention.

Thus, in one aspect the present invention relates to a method for selective breeding, comprising determining an estimated breeding value of a bovine subject using a method as defined herein for determining a genomic estimated breeding value, using said bovine subject as sire or dam for breeding if the estimated breeding value of said bovine subject is larger than, equal to, or differs less than a predetermined amount from a desired breeding value for the bovine subjects and/or the offspring. The genomic estimated breeding value may be determined without inclusion of phenotypic registrations of the bovine subjects or its relatives, and thus, the method also applies to the use of a bovine subjects as sire or dam before/prior to an udder health, fertility and/or other health phenotype associated with the estimated breeding value becomes manifest. For the same reason, the method also applies to the use of the bovine subject as sire or dam, when that bovine subject does not have any offspring and/or any phenotypic records of its offspring or other relatives.

According to the method of the present invention for selective breeding, the bovine subject is used as sire or dam for breeding if its genomic estimated breeding value is larger than, equal to, or differs less than a predetermined amount from a desired breeding value for the bovine subject or the offspring. The desired breeding value for the bovine subject or the offspring is apparent for those skilled in the art, and the tolerance with respect to predetermined difference between the breeding values of the parent and offspring are also within the skills of the trained practitioner. The desired breeding value and the tolerance with in terms of the accepted difference from the predetermined value depend on the phenotype with respect to which the breeding value is determined. In general a difference between the estimated breeding value and the desired breeding value of less than 20, such as less than 10, such as less than 9, for example less than 8, such as less than 7, for example less than 6, such as less than 5, for example less than 4, such as less than 3, for example less than 2, such as less than 1.

The breeding value is determined with respect to any phenotype as described herein. In preferred embodiments, the breeding value is determined in respect of udder health, fertility and/or other health, such as a health index comprising reproductive diseases, digestive diseases, feet and leg diseases. In a preferred embodiment, the breeding value is determined in respect of udder health, such as an udder health index comprising resistance to clinical mastitis and/or cell count.

### Sample

According to the present invention, a phenotypic trait according to the present invention is determined by detecting the absence or presence of a genetic marker allele or a specific combination of genetic marker alleles in a sample of any source comprising genetic material. Thus, detection of a genetic marker may be performed on samples selected from the group consisting of blood, semen (sperm), urine, liver tissue, muscle, skin, hair, follicles, ear, tail, fat, testicular tissue, lung tissue, saliva, spinal cord biopsy and any other tissue.

In preferred embodiments the sample is selected from the group consisting of blood, urine, skin, hair, ear, tail, liver and muscle. In another preferred embodiment the sample is selected from the group consisting of blood, liver tissue and muscle. In particularly preferred embodiments the sample is blood. In another particularly preferred embodiment the sample is liver tissue. In yet another particularly preferred embodiment the sample is muscle. In yet a further preferred embodiment the sample is blood and/or milk.

For genotyping, such as SNP and/or microsatellite genotyping, nucleic acid may be extracted from the samples by a variety of techniques. For example Genomic DNA may be isolated from the sample by treatment with proteinase K followed by extraction with phenol (Sambrook et al. 1989). However, the sample may also be used directly.

The amount of the nucleic acid used for microsatellite genotyping for detection of a genetic marker allele according to the method of the present invention is in the range of nanograms to micrograms. It is appreciated by the person skilled in the art that in practical terms no upper limit for the amount of nucleic acid to be analysed exists. The problem that the skilled person encounters is that the amount of sample to be analysed is limited. Therefore, it is beneficial that the method of the present invention can be performed on a small amount of sample and thus a limited amount of nucleic acid in the sample is required. The amount of the nucleic acid to be analysed is thus at least 1 ng, such as at least 10 ng, for example at least 25 ng, such as at least 50 ng, for example at least 75 ng, such as at least 100 ng, for example at least 125 ng, such as at least 150 ng, for example at least 200 ng, such as at least 225 ng, for example at least 250 ng, such as at least 275 ng, for example at least 300 ng, 400 ng, for example at least 500 ng, such as at least 600 ng, for example at least 700 ng, such as at least 800, ng, for example at least 900 ng or such as at least 1000 ng.

In one preferred embodiment the amount of nucleic acid as the starting material for the method of the present invention is 20-50 ng. In a specifically preferred embodiment, the starting material for the method of the present invention is at 30-40 ng.

### Detection

The method according to the present invention for determining a genotype for a plurality of genetic markers, a phenotype, and or a phenotypic trait according to the present invention of a bovine subject comprises detecting in a sample from said bovine subject the presence or absence of at least one genetic marker allele or a plurality genetic markers of the present invention. The genetic marker allele, plurality of markers or specific combination of genetic marker alleles is associated with said phenotypic trait of said bovine subject and/or offspring or other relatives therefrom. In a preferred embodiment, the genetic markers are selected from the group set out in the BovineSNP50 BeadChip from Illumine Inc. The genetic markers, or a complementary sequence as well as transciptional (mRNA) and translational products (polypeptides, proteins) therefrom may be identified by any method known to those of skill within the art.

It will be apparent to the person skilled in the art that there are a large number of analytical procedures which may be used to detect the presence or absence of variant nucleotides at one or more of positions mentioned herein in the specified region. Mutations or polymorphisms within or flanking the specified region can be detected by utilizing a number of techniques. Nucleic acid from any nucleated cell can be used as the starting point for such assay techniques, and may be isolated according to standard nucleic acid preparation procedures that are well known to those of skill in the art. In general, the detection of allelic variation requires a mutation discrimination technique, optionally an amplification reaction and a signal generation system.

A number of mutation detection techniques are listed below. Some of the methods listed are based on the polymerase chain reaction (PCR), wherein the method according to the present invention includes a step for amplification of the nucleotide sequence of interest in the presence of primers based on the nucleotide sequence of the variable nucleotide sequence. The methods may be used in combination with a number of signal generation systems, a selection of which is listed further below.

| | |
|---|---|
| General techniques | DNA sequencing, Sequencing by hybridisation, |
| | SNAPshot |
| Scanning techniques | Single-strand conformation polymorphism analysis, |
| | Denaturing gradient gel electrophoresis, Temperature |
| | gradient gel electrophoresis, Chemical mismatch |
| | cleavage, cleavage, heteroduplex analysis, enzymatic |
| | mismatch cleavage |
| Hybridisation based | Solid phase hybridisation: Dot blots, Multiple allele |
| techniques | specific diagnostic assay (MASDA), Reverse dot blots, |
| | Oligonucleotide arrays (DNA Chips) |
| | Solution phase hybridisation: Taqman -U.S. Pat. No. |
| | 5,210,015 & 5,487,972 (Hoffmann-La Roche), Molecular |
| | Beacons -- Tyagi et al (1996), Nature Biotechnology, 14, |
| | 303; WO 95/13399 (Public Health Inst., New York), |
| | Lightcycler, optionally in combination with Fluorescence |
| | resonance energy transfer (FRET). |
| Extension based | Amplification refractory mutation system (ARMS), |
| techniques | Amplification refractory mutation system linear extension |
| | (ALEX) - European Patent No. EP 332435 B1 (Zeneca |
| | Limited), Competitive oligonucleotide priming system |
| | (COPS) - Gibbs et al (1989), Nucleic Acids Research, 17, |
| | 2347. |
| Incorporation based | Mini-sequencing, Arrayed primer extension (APEX) |
| techniques | |
| Restriction Enzyme | Restriction fragment length polymorphism (RFLP), |
| based techniques | Restriction site generating PCR |
| Ligation based | Oligonucleotide ligation assay (OLA) |
| techniques | |
| Other | Invader assay |
| Various Signal | Fluorescence: |
| Generation or | Fluorescence resonance energy transfer (FRET), |
| Detection Systems | Fluorescence quenching, Fluorescence polarisation-- |
| | United Kingdom Patent No. 2228998 (Zeneca Limited) |
| Other | Chemiluminescence, Electrochemiluminescence, Raman, |
| | Radioactivity, Colorimetric, Hybridisation protection |
| | assay, Mass spectrometry |

Further amplification techniques are found elsewhere herein. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2nd Edition by Newton & Graham, BIOS Scientific Publishers Limited, 1997.

The detection of genetic marker alleles and/or combinations of genetic marker alleles can according to one embodiment of the present invention be achieved by a number of techniques known to the skilled person, including typing of microsatellites or short tandem repeats (STR), restriction fragment length polymorphisms (RFLP), detection of deletions or insertions, random amplified polymorphic DNA (RAPIDs) or the typing of single nucleotide polymorphisms by methods such as restriction fragment length polymerase chain reaction, allele-specific oligomer hybridisation, oligomer-specific ligation assays, hybridisation with PNA or locked nucleic acids (LNA) probes.

| | |
|---|---|
| Further amplification | Self sustained replication (SSR), |
| techniques | Nucleic acid sequence based |
| | amplification (NASBA), |
| | Ligase chain reaction (LCR), |
| | Strand displacement amplification (SDA) |

A primer of the present invention is a nucleic acid molecule sufficiently complementary to the sequence on which it is based and of sufficiently length to selectively hybridise to the corresponding region of a nucleic acid molecule intended to be amplified. The primer is able to prime the synthesis of the corresponding region of the intended nucleic acid molecule in the methods described above. Similarly, a probe of the present invention is a molecule for example a nucleic acid molecule of sufficient length and sufficiently complementary to the nucleic acid sequence of interest which selectively binds to the nucleic acid sequence of interest under high or low stringency conditions.

The genetic marker associated with a phenotypic trait according to the present invention can be detected by a number of methods known to those of skill within the art. For example, the genetic marker may be identified by genotyping using a method selected from the group consisting of single nucleotide polymorphisms (SNPs), microsatellite markers, restriction fragment length polymorphisms (RFLPs), DNA chips, amplified fragment length polymorphisms (AFLPs), randomly amplified polymorphic sequences (RAPDs), sequence characterised amplified regions (SCARs), cleaved amplified polymorphic sequences (CAPSs), nucleic acid sequencing, and microsatellite genotyping.

In a one embodiment, the genetic marker allele or combination of alleles associated with a phenotypic trait according to the present invention is detected by microsatellite genotyping. Microsatellite genotyping may be performed by amplification of the microsatellite marker by sequence specific oligonucleotide primers, and subsequent analysis of the amplification product, in terms of for example length, quantity and/or sequence of the amplification product.

In a preferred embodiment, the genetic markers of the present invention are genotyped, i.e. the genetic marker alleles of the methods and products of the present invention are determined, by gene chip technology/DNA microarrays. In a more preferred embodiment, the genetic marker allele, plurality of genetic marker alleles or combination or plurality of alleles are determined by a DNA array, such as the BovineSNP50 BeadChip, which is a multi-sample genotyping panel powered by Illumina's Infinium® II Assay.

In another preferred embodiment, the genetic marker allele, plurality of genetic marker alleles, or combination or plurality of alleles associated with a phenotype, or phenotypic trait according to the present invention is detected by a DNA array, such as the BovineSNP50 BeadChip, which is a multi-sample genotyping panel powered by Illumina's Infinium® II Assay provided by Illumina Inc.

### Kit and Computer Products

In one aspect, the present invention relates to a diagnostic kit for detecting the presence or absence in a bovine subject of at least one genetic marker allele as described herein. The kit will provide an easily applicable means of genotyping a bovine subject in respect of genetic marker alleles and/or combinations of genetic marker alleles of the genomic prediction model.

Specifically, the diagnostic kit is suitable for detection of the presence or absence of at least one genetic marker allele and/or combination of marker alleles, which is associated with at least one phenotypic trait of said bovine subject and/or offspring or other relatives therefrom. Examples of specific traits are provided elsewhere herein.

In one aspect, the kit of the present invention comprises means for detecting a plurality of genetic marker alleles, and/or a computer program and/or a computer readable medium as defined elsewhere herein.

Genotyping of a bovine subject in order to establish the genetic determinants of a phenotypic trait according to the present invention for that subject according to the present invention can be based on the analysis of genomic DNA which can be provided using standard DNA extraction methods as described herein. The genomic DNA may be isolated and amplified using standard techniques such as the polymerase chain reaction using oligonucleotide primers corresponding (complementary) to the polymorphic marker regions. Additional steps of purifying the DNA prior to amplification reaction may be included.

In one embodiment of the present invention, the kit comprises components for genotyping a bovine subject. Methods for genotyping are disclosed elsewhere herein. In a preferred embodiment, genotyping is SNP or microsatellite genotyping. Thus, the kit may comprise various components for performing SNP or microsatellite genotyping. For example, the kit may comprise at least one oligonucleotide for genotyping of a bovine subject. In a specific embodiment, the kit comprises at least one oligonucleotide for detecting a genetic marker allele selected from the group of genetic markers set out in the BovineSNP50 BeadChip from Illumine Inc.

Furthermore, the kit according to the present invention may comprise reagents and buffers required for genotyping. The exact composition of buffers and reagents depend on the method used for genotyping. In one embodiment, the kit comprises buffers required for amplification of DNA. In a particular embodiment, the kit comprises components for purification of DNA.

The diagnostic kit according to the present invention may further comprise at least one reference sample. The reference sample serves to verify that the genetic marker is correctly detected in the sample. Thus, the presence or absence of a genetic marker according to the present invention can be detected in parallel in the sample and the reference sample. The reference sample may either be a negative control, which does not comprise genetic material comprising a genetic marker allele or a combination of alleles according to the present invention, or the reference sample may be a positive control, which comprises genetic material comprising a genetic marker allele or combination of alleles according to the present invention. The reference sample thus serves to verify that the kit is used correctly. In one embodiment, the reference sample comprises an oligonucleotide sequence of an SNP marker allele associated with at least one trait as defined elsewhere herein. In a specific embodiment, the reference sample comprises an SNP marker or microsatellite marker oligonucleotide sequence associated with a specific phenotype, as defined elsewhere herein. In another specific embodiment, the reference sample comprises an NSP or microsatellite marker oligonucleotide sequence associated with a specific phenotypic trait, as defined elsewhere herein.

The kit according to the present invention may be provided with instructions for the performance of the detection method of the kit, and for the interpretation of the results.

The individual effect of a plurality of genetic marker alleles on a phenotype, such as udder health, fertility and/or other health, or the corresponding estimated breeding value (e.g. for udder health or another phenotype mentioned herein) of a bovine subject, determined according to the present invention is in one embodiment stored in a suitable media, such as in a non-volatile memory, such as a computer memory and/or a database. The suitable media may be located distantly from the location of the bovine subject for which a genomic estimated breeding value is determined according to the present invention.

The methods of the present invention may be embodied in a computer program product including program code portions for performing, when run on a programmable apparatus, a method according to the present invention, for example a method for determining the individual effect of a plurality of genetic marker alleles on a breeding value of one or more reference bovine subjects, or a method of estimating a breeding value of a bovine subject based on the genotype of said bovine subject for a plurality of genetic markers, said methods as described in more detail elsewhere herein. The present invention also in one aspect relates to a computer readable medium comprising data representing a computer program product as defined above. Thus, data representing the computer program product may be stored on a computer readable product, comprising, but not limited to, storage media such as magnetic storage media (ROMs, RAMs, floppy discs, magnetic tapes, etc.), optically readable storage media (CD-ROMs, DVDs, etc.), and carrier waves (transmission via the internet). Further, the computer program product may be implemented in a distributed fashion, e.g. comprising a first portion, e.g. for performing the method for determining the individual effect of a plurality of genetic marker alleles on a breeding value, and a second portion, e.g. for performing the method of estimating a genomic breeding value of a bovine subject, wherein the first and second portions may be arranged to be run on mutually different programmable apparatus and/or at mutually different (remote) locations.

One aspect of the present invention relates to a computer, a computer system and/or a programmable apparatus for performing a method of the present invention, said computer, a computer system and/or a programmable apparatus comprising a computer program product as and/or computer readable medium as described above.

Any use of a product of the present invention are inherently within the scope of the present invention. In particular, the present invention relates to the use of a computer program product, a computer readable medium, computer system and/or a programmable apparatus, and/or kit of the present invention for estimating a breeding value for a specific phenotype of a bovine subject, or for use in selective breeding.

### Granddaughter Design

The granddaughter design includes analysing data from DNA-based markers for grandsires that have been used extensively in breeding and for sons of grandsires where the sons have produced offspring. The phenotypic data that are to be used together with the DNA-marker data are derived from the daughters of the sons. Such phenotypic data could be for example milk production features, features relating to calving, fertility, meat quality, or disease. One group of daughters has inherited one allele from their father whereas a second group of daughters has inherited the other allele from their father. By comparing data from the two groups, information can be gained whether a fragment of a particular chromosome is harbouring one or more genes that affect the trait in question. It may be concluded whether a Quantitative trait loci is present within this fragment of the chromosome.

A prerequisite for performing a granddaughter design is the availability of detailed phenotypic data. In the present invention such data have been available (http://www.lr.dk/kvaeg/diverse/principles.pdf).

In contrast, genetic marker alleles and/or combinations of genetic marker alleles can be used directly to provide information of the traits passed on from parents to one or more of their offspring when a number of DNA markers on a chromosome have been determined for one or both parents and their offspring. The markers may be used to calculate the genetic history of the chromosome linked to the DNA markers.

### Frequency of Recombination

The frequency of recombination is the likelihood that a recombination event will occur between two genes or two markers. The frequency of recombination may be calculated as the genetic distance between the two genes or the two markers. Genetic distance is measured in units of centiMorgan (cM). One centiMorgan is equal to a 1% chance that a marker at one genetic locus will be separated from a marker at a second locus due to crossing over in a single generation. One centiMorgan is equivalent, on average, to one million base pairs.

In order to detect whether the genetic marker is present in the genetic material, standard methods well known to persons skilled in the art may be applied, for example by the use of nucleic acid amplification. In order to determine whether the genetic marker allele is genetically linked to a phenotypic trait according to the present invention, a permutation test can be applied when the regression method is used (Doerge and Churchill, 1996). The principle of the permutation test is well described by Doerge and Churchill (1996). A threshold at the 5% chromosome wide level is considered to be significant evidence for linkage between the genetic marker and a phenotypic trait according to the present invention.

The frequency of recombination is the likelihood that a recombination event will occur between two genes or two markers. The frequency of recombination may be calculated as the genetic distance between the two genes or the two markers. Genetic distance is measured in units of centiMorgan (cM). One centiMorgan is the length of chromosome wherein there is on average 0.01 cross-over per meiosis. If an uneven number of cross-overs occurs between two genetic markers, then a marker at one genetic locus will be separated from a marker at a second locus due to crossing over in a single generation. One centiMorgan is equivalent, on average, to one million base pairs. The relative position of a genetic marker, for example a microsatellite marker, may be designated in Morgan or centiMorgan with reference to its distance from a proximal position in the chromosome located at 0 cM.

It is an object of the present invention to provide genetic marker alleles or a specific combination of such marker alleles that are associated with a trait as defined herein. The specific genetic marker allele can be identified according to the present invention, by detecting a genetic marker, such as an SNP marker, which is genetically coupled to said first genetic marker allele.

### EXAMPLES

### Example 1

### Genomic Prediction 1

### Materials and Methods

### Data

2000 Holstein bulls (covering birth year from 1973 to 2002) were chosen to be genotyped for 50 k SNP loci. After the editing, the number of SNP loci reduced to 38055, and the number of typed bulls reduced to 1898, among which 1481 bulls were born during 1993-2002. EBVs (index) from current genetic evaluation were used as response variable to estimate SNP effect. In total 17 single or complex traits were analyzed in this study.

### Statistical Model

A Bayesian Gibbs sampling approach was applied to estimate SNP effect using a simple model,

yᵢ=μ+Σⱼ₌₁^{m}(qᵢⱼ₁+qᵢⱼ₂)vⱼ+eᵢ

where yᵢ is pedigree based EBV of individual i, μ is the intercept, m is the number of SNP loci, qᵢⱼ₁ and qᵢⱼ₂ are the scaled effects of paternal and maternal SNPs at locus j, vⱼ (vⱼ>0) is the scale factor (standard deviation) for qⱼₖ at locus j, and eᵢ is the random residual.

It is specified that the prior distribution of qⱼₖ is a standard normal distribution, i.e.,

q˜N(0,I)

Scale factors vⱼ's were assumed to have either a common prior distribution

v˜TN(0,Iσ²_{SNP}), v>0

or a mixture prior distribution,

v₀˜TN(0,I₀σ²ᵥ₀) v₀>0, v₁˜TN(0, I₁σ²ᵥ₁) v₁>0

The prior distribution of μ, σ²ᵥ and σ²ᵥ₁ were assumed to be improper uniform distribution, while σ²ᵥ₀ was set with a small value. In this study, σ²ᵥ₀ was set to 0.01 in all traits.

### Cross Validation

The accuracy of GEBV was evaluated by a five-fold cross validation. Thus, five subsets were created from the whole data, each left two year's records out (subset 1 without bulls which born in 1993 and 1994, subset 2 without 1995 and 1996, and so on). Each of the five subsets was used as training data to estimated SNP effect, and the corresponding “leave out” data as test data to predict GEBV (Table 1). The five test datasets comprised a total test data including 1481 bulls.

TABLE 1
Data structure in the five-fold cross validation

| Valid | Training data | | Test data | |
|---|---|---|---|---|
| Dataset | Data derivation | No. records | Data derivation | No. records |
| 1 | Ex 1993-1994 | 1704 | 1993-1994 | 194 |
| 2 | Ex 1995-1996 | 1591 | 1995-1996 | 307 |
| 3 | Ex 1997-1998 | 1575 | 1997-1998 | 323 |
| 4 | Ex 1999-2000 | 1549 | 1999-2000 | 349 |
| 5 | Ex 2001-2002 | 1590 | 2001-2002 | 308 |

Pilot analysis: First, a pilot analysis on four traits (protein yield, combined yield, udder, and female fertility) was carried out to find appropriate prior distribution of scale factors (v). The analysis included five scenarios: mixture priors with π₁=5%, 10%, 20%, 50% and a common prior for all loci.

Final analysis: The most appropriate (best prediction) prior distribution (which was common prior distribution according to the pilot study) was used to analyse all the 17 traits.

### Results

TABLE 2
Correlation between EBV and GEBV from models using different
priors for scale factors (standard deviation) of SNP effects.

| Trait | P05 | P10 | P20 | P50 | Common |
|---|---|---|---|---|---|
| Fertility | 0.575 | 0.614 | 0.638 | 0.648 | 0.645 |
| Protein yield | 0.486 | 0.537 | 0.579 | 0.601 | 0.603 |
| Udder health | 0.485 | 0.517 | 0.551 | 0.572 | 0.572 |
| Yield index | 0.459 | 0.503 | 0.547 | 0.573 | 0.577 |

TABLE 3
Cross-year R² between EBV and GEBV for different groups:
all bulls in the test data (All), bulls with reliability
of EBV for yield equal to or higher than 93% (SH93),
94% (SH94), 95% (SH95)and 96% (SH96).

| | All | SH93 | SH94 | SH95 | SH96 |
|---|---|---|---|---|---|
| Trait | n = 1481 | N = 886 | n = 484 | N = 198 | n = 103 |
| Birth ease | 0.365 | 0.366 | 0.380 | 0.441 | 0.481 |
| Body score | 0.236 | 0.231 | 0.224 | 0.245 | 0.324 |
| Calv. ease | 0.360 | 0.381 | 0.399 | 0.382 | 0.413 |
| Fat | 0.503 | 0.544 | 0.583 | 0.598 | 0.599 |
| Fat percent | 0.691 | 0.696 | 0.718 | 0.746 | 0.802 |
| Fertility | 0.448 | 0.441 | 0.442 | 0.481 | 0.632 |
| Health | 0.328 | 0.352 | 0.366 | 0.337 | 0.394 |
| Leg | 0.425 | 0.413 | 0.433 | 0.512 | 0.527 |
| Longevity | 0.250 | 0.250 | 0.277 | 0.413 | 0.536 |
| Milk | 0.489 | 0.509 | 0.526 | 0.564 | 0.595 |
| Milk organ | 0.368 | 0.370 | 0.392 | 0.390 | 0.430 |
| Milk speed | 0.397 | 0.401 | 0.410 | 0.411 | 0.442 |
| Protein | 0.498 | 0.517 | 0.543 | 0.565 | 0.544 |
| Prot. percent. | 0.491 | 0.489 | 0.521 | 0.519 | 0.618 |
| Temperament | 0.375 | 0.379 | 0.423 | 0.489 | 0.505 |
| Udder health | 0.330 | 0.362 | 0.391 | 0.420 | 0.515 |
| Yield | 0.479 | 0.504 | 0.538 | 0.544 | 0.518 |
| Average | 0.414 | 0.424 | 0.445 | 0.474 | 0.522 |

TABLE 4
Within-year R² between EBV and GEBV for different groups:
all bulls in the test data (All), bulls with reliability
of EBV for yield equal to or higher than 93% (SH93),
94% (SH94), 95% (SH95)and 96% (SH96).

| | All | SH93 | SH94 | SH95 | SH96 |
|---|---|---|---|---|---|
| Trait | n = 1481 | N = 886 | n = 484 | N = 198 | n = 103 |
| Birth ease | 0.363 | 0.365 | 0.380 | 0.427 | 0.474 |
| Body score | 0.206 | 0.210 | 0.206 | 0.235 | 0.287 |
| Calv. ease | 0.342 | 0.373 | 0.398 | 0.387 | 0.457 |
| Fat | 0.425 | 0.470 | 0.530 | 0.583 | 0.625 |
| Fat percent | 0.688 | 0.694 | 0.716 | 0.733 | 0.799 |
| Fertility | 0.416 | 0.412 | 0.433 | 0.473 | 0.640 |
| Health | 0.292 | 0.332 | 0.352 | 0.362 | 0.447 |
| Leg | 0.404 | 0.392 | 0.420 | 0.496 | 0.534 |
| Longevity | 0.222 | 0.238 | 0.271 | 0.409 | 0.522 |
| Milk | 0.419 | 0.449 | 0.489 | 0.545 | 0.594 |
| Milk organ | 0.315 | 0.338 | 0.367 | 0.397 | 0.457 |
| Milk speed | 0.384 | 0.395 | 0.401 | 0.401 | 0.423 |
| Protein | 0.364 | 0.406 | 0.471 | 0.551 | 0.537 |
| Prot. percent. | 0.479 | 0.477 | 0.508 | 0.515 | 0.600 |
| Temperament | 0.348 | 0.356 | 0.408 | 0.461 | 0.433 |
| Udder health | 0.327 | 0.368 | 0.401 | 0.459 | 0.540 |
| Yield | 0.333 | 0.379 | 0.454 | 0.530 | 0.514 |
| Average | 0.372 | 0.391 | 0.424 | 0.468 | 0.522 |

TABLE 5
Reliability of GEBV for different groups: all bulls in the test
data (All), bulls with reliability of EBV for yield equal to or
higher than 93% (SH93), 94% (SH94), 95% (SH95)and 96% (SH96).

| | All | SH93 | SH94 | SH95 | SH96 |
|---|---|---|---|---|---|
| Trait | n = 1481 | N = 886 | n = 484 | N = 198 | n = 103 |
| Birth ease | 0.460 | 0.465 | 0.480 | 0.490 | 0.555 |
| Body score | 0.492 | 0.495 | 0.509 | 0.520 | 0.580 |
| Calv. ease | 0.428 | 0.433 | 0.449 | 0.462 | 0.529 |
| Fat | 0.602 | 0.605 | 0.617 | 0.625 | 0.674 |
| Fat percent | 0.681 | 0.684 | 0.693 | 0.698 | 0.737 |
| Fertility | 0.632 | 0.635 | 0.645 | 0.652 | 0.698 |
| Health | 0.487 | 0.491 | 0.506 | 0.520 | 0.579 |
| Leg | 0.524 | 0.528 | 0.540 | 0.550 | 0.605 |
| Longevity | 0.407 | 0.409 | 0.424 | 0.434 | 0.498 |
| Milk | 0.626 | 0.629 | 0.640 | 0.648 | 0.695 |
| Milk organ | 0.462 | 0.466 | 0.480 | 0.493 | 0.559 |
| Milk speed | 0.451 | 0.455 | 0.471 | 0.483 | 0.550 |
| Protein | 0.673 | 0.676 | 0.685 | 0.692 | 0.734 |
| Prot. percent. | 0.497 | 0.501 | 0.515 | 0.525 | 0.588 |
| Temperament | 0.488 | 0.492 | 0.507 | 0.518 | 0.580 |
| Udder health | 0.438 | 0.442 | 0.457 | 0.469 | 0.536 |
| Yield | 0.660 | 0.663 | 0.673 | 0.680 | 0.723 |
| Average | 0.530 | 0.533 | 0.547 | 0.557 | 0.613 |

Reliability = 1-Se²/Vg, where Se is posterior standard deviation of GEBV, Vg is genomic variance estimated from data.

### Example 2

### Genomic Prediction 2

### Reference Data for Estimating SNP Effect for Genomic Prediction in Our Study

Holstein bulls from 258 half-sib families (1-41 bulls each), born during years from 1986 to 2004 were genotyped using Illumina Bovine SNP50 BeadChip (Illumina, San Diego, Calif.). The marker data were edited using the following criteria: 1) the locus was deleted if the minor allele frequency less than 5%, or the proportion of animals called for a genotype at this locus was less than 95%, or the average GenCall score at the locus was less than 0.65; 2) the individual was deleted if the call rate was less than a score of 0.85; 3) a marker type for an individual had a GenCall score less than 0.6. After the editing, there were 3,330 bulls and 38,134 SNP (single nucleotide polymorphism) markers available.

Published conventional EBV were used as response variables to estimate SNP effects. The EBV and their reliability for the genotyped bulls were obtained from official evaluations in April 2009.

### Statistical Model for Genomic Prediction

Several statistical models and algorithms are suitable for predicting breeding values based on dense markers, for example BLUP, BayesA and BayesB can be used to analyze simulated data and real data. A linear BLUP approach assumes that effects of all SNP are normal distributed with same variance. BayesA and BayseB (Meuwissen et al., 2001) allow each marker to have its own variances of allele effects. BayesB also models that most SNP have zero effect, but a few have moderate to large effects.

In this example, a Bayesian method, which captures the features of BayesA and BayesB but simplifies the computing algorithm, was used to estimate marker effects for genomic prediction. The model is:

$$y = 1 μ + {\sum }_{i = 1}^{m} {X}_{i} {q}_{i} {v}_{i} + e ,$$

where y is published conventional EBV, μ is the intercept, m is the number of SNP markers, qᵢ is the vector of scaled SNP effects (scaled by standard deviation) of marker with qᵢ˜N(0,I), vᵢ (vᵢ>0) is a scaling factor (standard deviation) for SNP effects of marker i, and e is the vector of residual. The effects of SNP types of marker i are the products of vᵢ and qᵢ.

Scaling factors vᵢ were assumed to have either a common prior distribution or a mixture prior distribution. A common prior distribution across the variances of chromosome segment effects, which leads to a slight or moderate differentiation between small and large effects of markers, was assumed to be a positive half-normal distribution,

vᵢ˜TN(0,σᵥ²), vᵢ>0

Mixture prior distribution, which lead to a strong differentiation between small and large effects of markers, assume that a proportion (p₀, typically large) of markers have a very small effect, and a proportion (p₁, typically small) of markers have a moderate or large effect. This was achieved by assuming that the prior distribution of vi was sampled from either a positive half-normal distribution with a small variance (σᵥ₀²) or a positive half-normal distribution with large variance (σᵥ₁²)

vᵢ˜π₀TN(0,σᵥ₀²)+π₁TN(σᵥ₁²)

The GEBV for individual k was defined as the sum of predicted effects of SNP over all markers

$${GEBV}_{k} = \hat{μ} + {\sum }_{i = 1}^{m} {x}_{i ( k . )} {q}_{i} {v}_{i}$$

A study on model validation shows that the common prior model performed generally better than the mixture prior models, therefore this model was used to estimate SNP effect for genomic prediction.

### Reliability of Genomic Estimated Breeding Value

The accuracy of GEBV were evaluated using a 5-fold cross validation. In each validation dataset, many half-sibs (about 500 individuals) were left out as test data, and the remaining as reference data. Two criteria were used to assess accuracy of genomic predictions. One was squared the correlation between GEBV and published conventional EBV in test datasets, where both GEBV and EBV were adjusted for birth-year mean to account for genetic trend, i.e., within-year squared correlation. The other was expected genomic reliability, obtained from prediction error variance (PEV) which was measured as posterior variance of each GEBV. To avoid strong dependencies between test data and training data, the sires which had sons or grandsons in the training data were excluded from the validation.

Squared correlation between GEBV and EBV (r²GEBV,EBV) for animals in the test data and expected reliability of GEBV

| Trait | r²_{GEBV, EBV} | Expected reliability |
|---|---|---|
| Fertility | 0.412 | 0.566 |
| Other-health | 0.426 | 0.593 |
| Udder-health | 0.435 | 0.557 |

r²GEBV,EBV were lower than expected reliabilities. The lower r²GEBV,EBV could be due to the facts that EBV contained error and the animals in the validation were selected from elite parents, instead of random samples. The reliability in the above table is calculated from the animals in test data which were not used to estimated SNP effect. It means the reliability of GEBV is for those animals which do not have their own record (no progeny records for bull).

For young bulls without progeny records, their EBV can be obtained from parent average EBV. The reliability of parent average EBV is about half of the reliability of GEBV in this study, indicating genomic prediction is considerably better than conventional prediction of young bull.

### Example 3

Breeding values for 30 sires where calculated for fertility index, udder health index and other health index (health index comprising reproductive diseases, digestive diseases and/or feet and leg diseases) phenotypes. Conventional EBVs were calculated from parent EBV (PA), and EBVs were calculated, which include records of progeny tests. Moreover, GEBV were calculated on the basis of the genotype of the sire for a plurality of genetic SNP marker, without including records of progeny test. The bulls were genotyped and GEBV calculated as described in example 2. The values are listed in table 2. It is clear that predicted GEBV are more similar to the EBV after progeny test than parent average EBV. Thus, GEBV predicted according to the present invention is a more reliable tool than PA EBV for predicting the genetic merit of a bull, in the absence of progeny records.

TABLE 2
EBV from Parent average (PA), GEBV and EBV
after progeny test for a group of bulls.

| | Fertility | | | Other health | | | Udder-health | | |
|---|---|---|---|---|---|---|---|---|---|
| id | PA | GEBV | EBV | PA | GEBV | EBV | PA | GEBV | EBV |
| 1 | 97 | 105 | 103 | 103 | 110 | 120 | 93 | 105 | 114 |
| 2 | 90 | 81 | 67 | 86 | 89 | 98 | 99 | 99 | 97 |
| 3 | 93 | 89 | 80 | 97 | 101 | 111 | 97 | 92 | 82 |
| 4 | 92 | 98 | 103 | 95 | 104 | 106 | 102 | 108 | 108 |
| 5 | 103 | 109 | 110 | 108 | 112 | 108 | 112 | 118 | 126 |
| 6 | 95 | 96 | 101 | 91 | 96 | 104 | 97 | 103 | 103 |
| 7 | 103 | 99 | 98 | 102 | 107 | 109 | 94 | 94 | 85 |
| 8 | 96 | 88 | 83 | 93 | 95 | 91 | 92 | 85 | 81 |
| 9 | 106 | 102 | 97 | 107 | 105 | 100 | 107 | 100 | 101 |
| 10 | 103 | 102 | 100 | 102 | 107 | 117 | 94 | 88 | 89 |
| 11 | 90 | 90 | 91 | 86 | 85 | 79 | 99 | 106 | 109 |
| 12 | 102 | 94 | 92 | 102 | 98 | 106 | 92 | 86 | 87 |
| 13 | 88 | 90 | 90 | 97 | 100 | 101 | 97 | 99 | 99 |
| 14 | 104 | 104 | 95 | 97 | 98 | 101 | 95 | 107 | 110 |
| 15 | 106 | 110 | 114 | 105 | 104 | 98 | 104 | 101 | 101 |
| 16 | 100 | 101 | 96 | 102 | 96 | 93 | 94 | 92 | 92 |
| 17 | 92 | 96 | 105 | 100 | 103 | 103 | 100 | 106 | 118 |
| 18 | 100 | 106 | 107 | 96 | 95 | 92 | 88 | 97 | 99 |
| 19 | 106 | 105 | 108 | 102 | 96 | 98 | 103 | 95 | 97 |
| 20 | 93 | 96 | 84 | 93 | 86 | 81 | 101 | 94 | 90 |
| 21 | 110 | 94 | 90 | 111 | 110 | 107 | 110 | 107 | 108 |
| 22 | 101 | 94 | 90 | 103 | 102 | 102 | 114 | 119 | 123 |
| 23 | 98 | 93 | 89 | 95 | 99 | 102 | 100 | 98 | 96 |
| 24 | 93 | 91 | 93 | 94 | 99 | 102 | 96 | 101 | 111 |
| 25 | 93 | 90 | 88 | 101 | 99 | 109 | 95 | 85 | 84 |
| 26 | 101 | 103 | 98 | 98 | 105 | 105 | 105 | 101 | 98 |
| 27 | 98 | 95 | 92 | 90 | 94 | 95 | 98 | 93 | 86 |
| 28 | 104 | 100 | 99 | 98 | 100 | 97 | 99 | 92 | 92 |
| 29 | 95 | 95 | 98 | 97 | 95 | 94 | 92 | 89 | 89 |
| 30 | 99 | 105 | 108 | 98 | 92 | 88 | 100 | 103 | 98 |

### Example 4

### Reliability of Genomic Estimated Breeding Values in the Danish Holstein Population

This example evaluates the reliability of genomic estimated breeding values (GEBV) in the Danish Holstein population. The data in the analysis included 3,330 bulls with both published conventional EBV and single nucleotide polymorphism (SNP) markers. After data editing, 38,134 SNP markers were available. In the analysis, all SNPs were fitted simultaneously as random effects in a Bayesian variable selection model which allows heterogeneous variances for different SNP markers. The response variables were the official EBV. Direct GEBV were calculated as the sum of individual SNP effects. Initial analyses of 4 index traits were carried out to compare models with different intensities of shrinkage for SNP effects, i.e., mixture prior distributions of scaling factors (standard deviation of SNP effects) assuming 5%, 10%, 20% or 50% of SNP having large effects and the others having very small or no effects, and a single prior distribution common for all SNP. It was found that in general the model with a common prior distribution of scaling factors had better predictive ability than any mixture prior models. Therefore, a common prior model was used to estimate SNP effects and breeding values for all 18 index traits. Reliability of GEBV was assessed by squared correlation between GEBV and conventional EBV, and expected reliability obtained from prediction error variance, using a 5-fold cross validation. Squared correlations between GEBV and published EBV (without any adjustment) ranged from 0.252 to 0.700 with an average of 0.418. Expected reliabilities ranged from 0.494 to 0.733 with an average of 0.546. The results show selection of bovine subjects for breeding based on GEBV can greatly improve the accuracy of selection for young bulls and bull dams, compared with traditional selection based on parent average.

### Materials and Methods

### Data

Holstein bulls from 258 half-sib families (1-41 bulls each), born during years from 1986 to 2004 were genotyped using Illumina Bovine SNP50 BeadChip (Illumina, San Diego, Calif.). The marker data were edited using the following criteria: 1) the locus was deleted if the minor allele frequency less than 5%, or the proportion of animals called for a genotype at this locus was less than 95%, or the average GenCall score at the locus was less than 0.65; 2) the individual was deleted if the call rate was less than a score of 0.85; 3) a marker type for an individual had a GenCall score less than 0.6. After the editing, there were 3,330 bulls and 38,134 SNP (single nucleotide polymorphism) markers available. In the analysis of SNP effects and genomic prediction, missing SNP at a particular marker in some animals was treated as an extra allele. It corresponded to replacing the effect of missing SNP at a marker with population mean of this marker.

Published conventional EBV were used as response variables to estimate SNP effects. The EBV and their reliability for the genotyped bulls were obtained from official evaluations in April 2009. In total 18 index traits were analyzed in this example. Except for fat percentage and protein percentage, the traits are the sub-traits in the new Nordic Total merit index (NTM). Detailed descriptions on these index traits and their EBV are given in Danish Cattle Federation (2006).

### Statistical Model

All individual SNP markers were used as predictors and conventional EBV (PA EBV) were used as response variables weighted by a function of reliability of EBV (see herein below for details). A Bayesian method which captures the features of BayesA and BayesB but simplifies the computing algorithm was used to estimate marker effects for genomic prediction. The method applies the methodology of variable selection presented by George and McCulloch (1993). A detailed description of the method was presented by Villumsen et al. (2009) and Meuwissen and Goddard (2004). The following model was used to fit EBV data:

$$y = 1 μ + {\sum }_{i = 1}^{m} {X}_{i} {q}_{i} {v}_{i} + e$$

where y is the vector of published conventional EBV, μ is the intercept, m is the number of SNP markers, qᵢ is the vector of scaled SNP effects (scaled by standard deviation) of marker i with qᵢ˜N(0,I), vᵢ (vᵢ>0) is a scaling factor (standard deviation) for SNP effects of marker i, and e is the vector of residual with e˜N(0,Iσₑ²). The effects of SNP alleles of marker i are the products of vᵢ and qᵢ. Scaling factors vᵢ were assumed to have either a common prior distribution or a mixture prior distribution. A common prior distribution across the variances of chromosome segment effects, which leads to a slight or moderate differentiation between small and large effects of markers, was assumed to be a positive half-normal distribution,

vᵢ˜TN(0,σᵥ²), vᵢ>0

Mixture prior distributions, which lead to strong differentiation between small and large effects of markers, assume that a proportion (π₀, typically large) of markers have very small effects, and another proportion (π₁=1−π₀, typically small) of markers have moderate or large effects. This was achieved by assuming that the prior distribution of vᵢ was sampled from either a positive half-normal distribution with a small variance (σᵥ₀²) or a positive half-normal distribution with large variance (σᵥ₁²),

vᵢ˜π₀TN(0,σᵥ₀²)+π₁TN(σᵥ₁²)

The prior distributions of μ, σᵥ² and σᵥ₁² were assumed to be improper uniform distributions, while σᵥ₀² was fixed at a small value. In this study, σᵥ₀² was set to 0.0001 for all traits. The genomic estimated breeding value (GEBV) for individual k was defined as the sum of predicted effects of SNP over all markers

$${GEBV}_{k} = \hat{μ} + {\sum }_{i = 1}^{m} {x}_{i ( k . )} {q}_{i} {v}_{i}$$

The effect of shrinkage intensity on accuracy of GEBV was investigated using 5 scenarios: 1) mixture prior of scaling factors with π₁=5%, 2) π₁=10%, 3) π₁=20%, 4) π₁=50%, and 5) common prior of scaling factors for all markers (i.e., π₁=100%).

An initial evaluation on fertility and udder-health using a common prior model was carried out to investigate the effect of weighting factor on accuracy of genomic prediction. It was found that a model using 1/(1−reliability of EBV) as weighting factor of response variable performed better than a model using reliability of EBV as weighting factor, the latter was better than a constant weight of 1 for all response variables (results not shown). Therefore weighting factor of 1/(1−reliability of EBV) was used in the further analysis. The weights were divided by the average weight to scale the weights to an average of 1. According to the definition, an EBV with reliability close to one would get an extremely high weight. To avoid possible problems due to extreme weights, reliabilities larger than 0.98 were replaced with 0.98 in the calculation of weight.

### Model Validation and Evaluation of Reliability of GEBV

The models with different priors for scaling factors and the accuracy of GEBV were evaluated using a 5-fold cross validation. In the cross validation, 134 half-sib families which have at least one bull born after 1993 were divided into 5 test datasets by the following procedures. First, the 134 half-sib families were assigned into 10 year classes (1994-2003) according to birth-year for the most of half-sibs. Then each two year classes formed a test dataset, i.e., 1994-1995 formed Test-set 1, 1996-1997 Test-set 2, and so on. The five test datasets comprised a total of 2,393 bulls. In each fold cross validation, the whole data excluded one test dataset to form a training dataset which was used to estimate marker effects and predict genomic breeding values of the “left out” animals. Detailed information on the whole data and 5 test datasets is shown in Table 11.

TABLE 11
Structures of the whole dataset and 5 test datasets

| | | No. of half-sib | Interval of birth |
|---|---|---|---|
| Dataset | No. of bulls | family | years |
| Whole | 3,330 | 258 | 1986-2004 |
| Test1 | 538 | 21 | 1989-1997 |
| Test2 | 469 | 20 | 1994-2000 |
| Test3 | 472 | 22 | 1997-2003 |
| Test4 | 458 | 29 | 1999-2004 |
| Test5 | 456 | 42 | 2001-2004 |

Two criteria were used to assess accuracy of genomic prediction. One was squared correlation between GEBV and published conventional EBV (r²_{GEBV,EBV}) in test datasets, where both GEBV and EBV were adjusted for birth-year mean to account for genetic trend, i.e., within-year squared correlation. The other was expected genomic reliability, obtained from prediction error variance (PEV) which was measured as posterior variance of each GEBV. To avoid strong dependencies between test data and training data, the sires which had sons or grandsons in the training data were excluded from the validation.

Five scenarios of prior distribution for scaling factors (standard deviations, vᵢ) of SNP effects were evaluated by analyzing 4 index traits (protein, fat percentage, udder-health, and female fertility). Model predictive ability was assessed by r²_{GEBV,EBV} in the 5-fold cross validation. The best model (which was a common prior distribution in this study) was used to analyze all the 18 index traits.

The analyses were carried out using the IBAY package (Janss Luc, Faculty of Agricultural Sciences, Aarhus University, Tjele, Denmark; personal communication). The Gibbs sampler was run as a single chain with a length of 50,000 samples. Convergence was monitored by graphical inspection in variance of scaling factors and the correlation between GEBV from two separate rounds. The first 20,000 samples were discarded as burn-in. Every 10^{th} sample of the remaining 30,000 was saved to estimate the features of the realized posterior distributions.

### Results

The mean, cross-year standard deviation and within-year standard deviation of the published EBV and their reliabilities for the genotyped bulls are shown in Table 2. The published EBV were standardized to a mean of 100 for the cows born 3-5 years (for production and conformation traits, animal model) or for the bulls born 7-9 years (for the remaining traits, sire model) before publication, and standardized to a standard deviation of 10 for bulls born in 1997 and 1998. The cross-year standard deviations for yield-index, protein, milk, fertility and other-disease were higher than 10, reflecting a genetic change over the years for these traits. Within-year standard deviations were close to 10 for all traits except for longevity (8.6) and growth (11.5), indicating that the genotyped bulls represented the genetic variation of bulls in the population. Reliabilities of EBV differed among 18 traits, and were consistent with heritabilities of the traits. There was a large variation in reliabilities within trait for low heritability traits, but small for high heritability traits.

TABLE 2
Mean, cross-year standard deviation (σ2t) and within-
year standard deviation (σ2w) of EBV and reliability
of EBV for the genotyped bulls

| | EBV | | | Reliability of EBV | | |
|---|---|---|---|---|---|---|
| Trait | Mean | σₜ² | σ_{w}² | Mean | σₜ² | σ_{w}² |
| Birth-index | 100.9 | 10.4 | 10.2 | 75.7 | 8.1 | 7.8 |
| Body-conf. | 97.3 | 10.9 | 10.2 | 81.1 | 6.5 | 6.1 |
| Calv-index | 99.5 | 10.1 | 9.9 | 70.5 | 8.5 | 7.9 |
| Fat | 98.8 | 11.2 | 9.4 | 93.4 | 2.9 | 2.5 |
| Fat % | 100.0 | 10.4 | 10.4 | 93.5 | 2.9 | 2.5 |
| Fertility | 104.6 | 10.9 | 9.9 | 69.1 | 10.5 | 8.7 |
| Other-disease | 102.0 | 10.6 | 9.9 | 61.2 | 12.3 | 10.5 |
| Feet-legs | 98.8 | 10.0 | 9.9 | 62.5 | 10.6 | 10.1 |
| Longevity | 100.4 | 8.5 | 8.4 | 61.7 | 7.8 | 6.8 |
| Milk | 98.9 | 12.6 | 10.6 | 93.4 | 2.9 | 2.5 |
| Udder-conf. | 97.2 | 10.1 | 9.5 | 77.9 | 7.6 | 7.1 |
| Milking-ability | 99.5 | 10.9 | 10.7 | 72.8 | 8.5 | 8.2 |
| Protein | 97.1 | 14.0 | 10.4 | 93.4 | 2.9 | 2.5 |
| Protein % | 98.2 | 10.3 | 10.2 | 93.5 | 2.9 | 2.5 |
| Temperament | 100.3 | 9.8 | 9.4 | 63.5 | 10.0 | 9.7 |
| Udder-health | 101.4 | 10.3 | 10.1 | 75.9 | 8.0 | 6.6 |
| Yield-index | 97.1 | 13.5 | 10.0 | 93.4 | 2.9 | 2.5 |
| Growth | 100.5 | 11.6 | 11.5 | 87.9 | 5.7 | 5.2 |
| Average | 99.6 | 10.9 | 10.0 | 78.9 | 6.7 | 6.1 |

### Influence of Prior Distribution on Genomic Prediction

The effect of changing the prior distribution of scaling factors on the predictive ability was investigated on fertility, protein, udder-health and fat percentage. The predictive abilities of the models with different priors for scaling factors were evaluated by within-year r²_{GEBV,EBV}, based on a 5-fold cross validation. Table 3 clearly shows that r²_{GEBV,EBV} increased with increasing prior proportion (π₁) of SNP with large effects within each subsets. Pooled over 5 subsets, r²_{GEBV,EBV} increased from 0.347 (π₁=0.05) to 0.412 (common prior, i.e., π₁=1.0) for fertility, from 0.279 to 0.412 for protein, from 0.338 to 0.435 for udder-health, and from 0.670 to 0.700 for fat percentage. r²_{GEBV,EBV} for fertility were similar when using models with π₁=0.50 and common prior, and for fat percentage, the values were similar when using models with π₁=0.20, π₁=0.50, and common prior. It was found that variation of r²_{GEBV,EBV} among the 5 subsets was larger in fertility and udder-health (the traits having low heritability) than protein and fat percentage (high heritability).

TABLE 3
Within-year squared correlation between GEBV and EBV, using
models with different prior distributions of scaling factors.

| | | Mixture | Mixture | Mixture | Mixture | |
|---|---|---|---|---|---|---|
| | | π₁ = | π₁ = | π₁ = | π₁ = | Common |
| Trait | Dataset | 5% | 10% | 20% | 50% | prior |
| Fertility | Test1 | 0.275 | 0.304 | 0.314 | 0.342 | 0.362 |
| | Test2 | 0.348 | 0.378 | 0.389 | 0.388 | 0.399 |
| | Test3 | 0.300 | 0.340 | 0.359 | 0.374 | 0.376 |
| | Test4 | 0.416 | 0.405 | 0.434 | 0.441 | 0.444 |
| | Test5 | 0.419 | 0.444 | 0.438 | 0.495 | 0.493 |
| | Pooled | 0.347 | 0.370 | 0.384 | 0.407 | 0.412 |
| Protein | Test1 | 0.284 | 0.315 | 0.357 | 0.393 | 0.401 |
| | Test2 | 0.304 | 0.363 | 0.405 | 0.371 | 0.413 |
| | Test3 | 0.283 | 0.331 | 0.354 | 0.374 | 0.375 |
| | Test4 | 0.283 | 0.352 | 0.392 | 0.407 | 0.438 |
| | Test5 | 0.233 | 0.309 | 0.368 | 0.410 | 0.420 |
| | Pooled | 0.279 | 0.337 | 0.378 | 0.394 | 0.412 |
| Udder-health | Test1 | 0.279 | 0.301 | 0.330 | 0.332 | 0.351 |
| | Test2 | 0.275 | 0.317 | 0.369 | 0.377 | 0.410 |
| | Test3 | 0.415 | 0.448 | 0.481 | 0.498 | 0.505 |
| | Test4 | 0.372 | 0.395 | 0.395 | 0.421 | 0.431 |
| | Test5 | 0.322 | 0.381 | 0.433 | 0.464 | 0.466 |
| | Pooled | 0.338 | 0.373 | 0.404 | 0.417 | 0.435 |
| Fat % | Test1 | 0.681 | 0.709 | 0.725 | 0.711 | 0.716 |
| | Test2 | 0.662 | 0.678 | 0.694 | 0.694 | 0.685 |
| | Test3 | 0.709 | 0.729 | 0.748 | 0.741 | 0.751 |
| | Test4 | 0.695 | 0.705 | 0.714 | 0.708 | 0.703 |
| | Test5 | 0.591 | 0.611 | 0.622 | 0.632 | 0.640 |
| | Pooled | 0.670 | 0.688 | 0.702 | 0.698 | 0.700 |

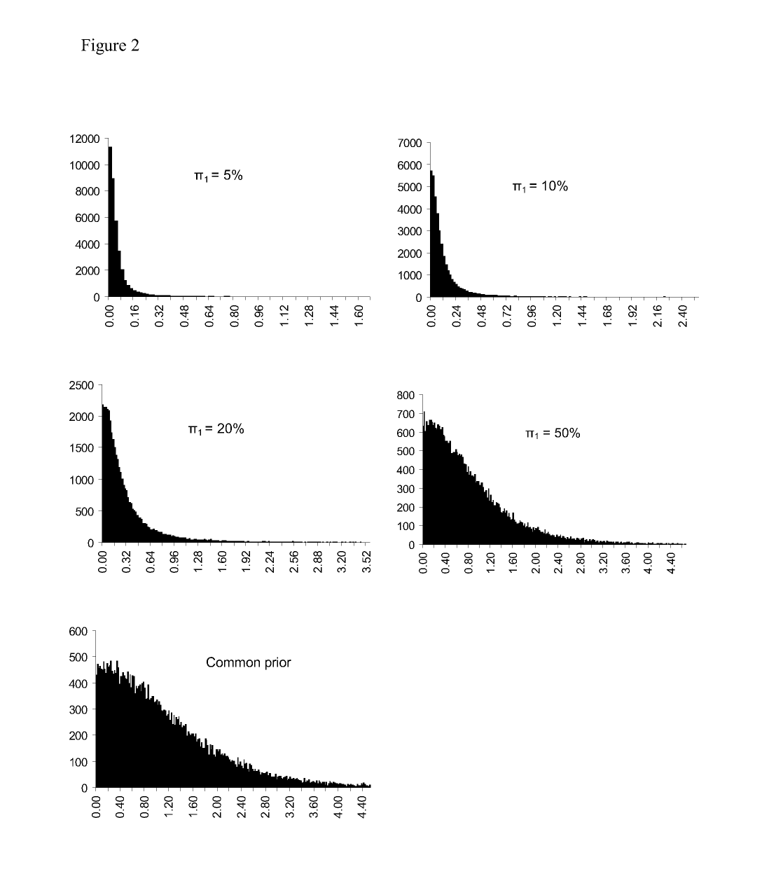

It was found that the prior distribution of scaling factors influenced the estimates of genetic marker effects. Taking fertility as an example, the distribution of marker effects (expressed as absolute value of the difference between two allele effects) followed a Gamma distribution for all scenarios (FIG. 2), when marker effects were measured as the difference between two alleles of a marker. With a prior that assumes a lower proportion (π₁) of marker with large effect (increasing the intensity of shrinkage), the distribution becomes more L shaped. The posterior percentages of the markers with an estimated effect less than 0.005 were 91%, 84%, 70%, 41% and 33% for π₁=0.05, 0.10, 0.20, 0.50, and a common prior, respectively.

It can be observed that the variation of marker effects increased with increasing shrinkage intensity (i.e., decreasing π₁) for all 4 traits. The means of marker effects decreased with increasing shrinkage intensity. Model fit was inspected by coefficient of determination (R² of the model based on the training data). As expected, the coefficient of determination increased with decreasing shrinkage intensity, due to increasing the freedom of explanatory variables to fit data.

### Reliabilities of GEBV

Since models with a common prior distribution of scaling factors generally provided better predictive abilities than mixture prior models, this model was chosen to estimate SNP effects and predict breeding values for 18 traits in Danish Holstein population. Table 4 presents within-year r²_{GEBV,EBV} and expected reliability of GEBV calculated from PEV for bulls in the test data. r²_{GEBV,EBV} ranged from 0.252 to 0.700 with an average of 0.418. Expected reliability ranged from 0.494 to 0.733 with an average of 0.546. For all traits, expected reliability was higher than r²_{GEBV,EBV}.

It was observed that the variation in r²_{GEBV,EBV} among the 18 traits was larger than the variation in expected reliabilities, but the patterns of ranks were similar. Product moment correlation and rank correlation between the two parameters were 0.883 and 0.813, respectively. Although the heritabilities for these traits differed considerably, the difference in accuracies of GEBV between low heritability traits and high heritability traits were relatively small, indicating that the reliability of GEBV was not very strongly influenced by heritability. For example, fertility, feet-legs, udder-health, and other-diseases had an expected reliability of GEBV and a r²_{GEBV,EBV} as high as or close to those for production traits.

TABLE 4
Within-year squared correlation between EBV and GEBV (r2GEBV,
EBV) and expected reliability of GEBV (calculated from prediction
error variance) for bulls in the test data.

| Trait | r²_{GEBV, EBV} | Expected reliability |
|---|---|---|
| Birth-index | 0.395 | 0.502 |
| Body-conf. | 0.252 | 0.499 |
| Calv-index | 0.369 | 0.525 |
| Fat | 0.487 | 0.569 |
| Fat % | 0.700 | 0.733 |
| Fertility | 0.412 | 0.566 |
| Other-disease | 0.426 | 0.593 |
| Feet-legs | 0.404 | 0.571 |
| Longevity | 0.317 | 0.494 |
| Milk | 0.481 | 0.562 |
| Udder-conf. | 0.395 | 0.511 |
| Milking-ability | 0.383 | 0.506 |
| Protein | 0.412 | 0.528 |
| Protein % | 0.518 | 0.559 |
| Temperament | 0.340 | 0.514 |
| Udder-health | 0.435 | 0.557 |
| Yield-index | 0.390 | 0.514 |
| Growth | 0.415 | 0.533 |
| Average | 0.418 | 0.546 |

### Discussion

The reliability of GEBV is a critical criterion in deciding whether GEBV is suitable for practical genetic evaluation. In the present example, 5 scenarios of prior distribution for variance of SNP effects were assessed. The common prior model performed generally better than the mixture prior models, therefore this model was used to investigate reliability of GEBV for 18 traits in Danish Holstein population. Cross validation shows that the accuracy of GEBV was significantly greater than conventional parent average EBV.

In the present example, reliability of GEBV was evaluated by r²_{GEBV,EBV} and expected reliability (calculated from PEV). It is seen that r²_{GEBV,EBV} were lower than the expected reliabilities. The lower r²_{GEBV,EBV} could be due to the facts that EBV contained error and the animals in the validation were selected from elite parents, instead of random samples. On the other hand, it is also possible that the expected reliability may overestimate the reliability of GEBV. An alternative is to measure reliability of GEBV as r²_{GEBV,EBV} divided by reliability of EBV. This is to assume the correlation between GEBV and EBV was through their correlation with true breeding value (i.e., no correlation between prediction errors of GEBV and EBV). Thus, r_{GEBV,EBV}=r_{GEBV,TBV}r_{EBV,TBV}, r²_{GEBV,TBV}=r²_{GEBV,EBV}/r²_{EBV,TBV}, where TBV is true breeding value. However, based on the present data, reliability estimated using this approach seemed too high to be acceptable for some low-heritability traits, thus implying that prediction errors of GEBV and EBV were not completely independent. We suggest that the true reliability of GEBV (r²_{GEBV,TBV}) in the present data could be between r²_{GEBV,EBV} and the expected reliability. Thus averaged over 18 traits, reliability of GEBV could be in the interval between 0.42 and 0.54. The figures are considerably greater than the reliability of the conventional parent average EBV. It indicates that genomic prediction can effectively improve the accuracy of pre-selection for young bulls, compared with traditional selection based on parent average.

The difference in reliability of GEBV between low heritability traits and high heritability traits was relatively small. In the present example, marker effects were estimated from published EBV. The influence of heritability on GEBV was through its influence on reliability of EBV. However the published EBVs were predicted from a very large dataset, resulting in a relatively high accuracy even for the traits with low heritability. Moreover, in genomic prediction, each individual in reference data has a contribution to marker effects. In other words, the GEBV of a candidate is actually obtained from the information of all individuals in the reference data. The benefit from information of other animals for the traits with low heritability is relatively greater than that for the traits with high heritability. The weak dependency on heritability indicates that genetic evaluation based on GEBV would be relatively more beneficial for the traits with low heritability. Previous studies on marker assisted selection have shown that gain in response rate is larger for traits with lower heritability (Lande and Thompson, 1990; Meuwissen and Goddard, 1996). However, these calculations were conditional on the fact that QTL had been identified, which is much more difficult for low heritability traits due to low statistical power of detection. Using genomic selection, the step of testing for QTL is circumvented. This is a reason that accurate GEBV can be obtained even for low heritability traits. As a consequence of a relatively weak dependency of GEBV on heritability, it becomes easier to improve functional traits and to obtain a balanced genetic progress between functional traits and production traits.

Five scenarios of prior distributions of the variance of SNP effects were investigated in this example. Using a single-marker approach, it was found that the model with a common distribution of scaling factors (standard deviations) had a better predictive ability than models assuming a mixture distribution. Also the predictive ability of the model using a mixture distribution increased with increasing assumed proportion of SNP having large effect. VanRaden et al. (2009) reported that predictive ability of a nonlinear BLUP model was considerably better than a linear BLUP model for fat percentage and protein percentage, while the predictive abilities were similar for other 25 traits. However, the linear BLUP model is not equivalent to the common prior model in the present study; the latter allows allele effects of different marks to have different variances. In simulation studies, Meuwissen et al. (2001) reported that the accuracy of GEBV using BayesB (similar to mixture prior distribution in the present example) was higher than that using BayesA (common prior distribution), and Lund et al. (2009) found that mixture models predicted breeding value better than the models with a common prior distribution of variances or the models with equal variance for all SNP. Both studies were based on the data in which QTL effects were simulated from a Gamma distribution with shape parameter 0.4 (L shape).

There are many possible reasons why the model with a mixture prior distribution of scaling factors did not perform better than the model with a common prior distribution in the present dataset. Firstly, the mixture prior distribution of scaling factors is based on the hypothesis that few genes have a large effect and a large number of genes have a small effect, and the distribution of QTL effects follows a Gamma distribution of L shape. The hypothesis is supported by the derived distribution of QTL effects reported by Hayes and Goddard (2001). However, the distribution of SNP effects is not necessary to be consistent with the distribution of QTL effects. Many SNP could located in a chromosome segment with large effect, thus the effect of the chromosome segment could be divided over many SNPs. On the other hand, the effect of a QTL might not be fully accounted for by a single marker, because of incomplete linkage between marker and QTL. In any way, for markers with small effect, it is difficult to find the optimal proportion and variance of scaling factors.

The accuracy of GEBV was evaluated using a 5-fold cross validation. The advantage of multiple-fold cross validation is that it can retain training data as large as possible, while keep the test data as large as required (with maximal total test data equal to the whole data). There was a variation in r²_{GEBV,EBV} between 5 sets of cross validations (Table 3), indicating that enough number of individuals in test data is important for validating reliability of GEBV. In the cross validation, each set of training data left many half-sib families out, instead of leaving a random sample out. This strategy greatly reduces the dependency between the training data and the test data, because the individuals in the test data did not have their sibs in the training data.

In this example, marker effects were estimated by fitting a model to published EBV. The advantage of using EBV is that they can be obtained directly from routine genetic evaluations. In addition they contain little random error, which greatly reduces the prediction error variance. This could be important in situations where the number of genotyped animals in the reference data is small. An alternative type of response variable is daughter yield deviation.

The reliabilities of GEBV in the present example indicate that genomic selection is a very promising tool in cattle breeding. Moreover, genomic prediction can be further improved by several approaches. Firstly, reliability of GEBV can increase with increasing data size (the number of individuals with both genotypes and phenotypes) to estimate marker effects. Secondly, the reliability may be improved by using other statistical models. Thirdly, the reliability of GEBV for an index trait may be improved by predicting genomic breeding value for each single trait, and then calculating the GEBV of the index trait, instead of predicting the index trait directly. Finally, higher accuracy of genomic selection can be obtained by a genomic selection index which combines GEBV and other sources of information, such as parent EBV from conventional national genetic evaluation.

### CONCLUSIONS

Averaged over all 18 index traits, the reliability of GEBV is considerably greater than the reliability of conventional parent average EBV. It clearly provides that genomic selection can greatly improve the accuracy of pre-selection for young bulls, compared with traditional selection based on parent average. Based on the data in this example, the model with a common prior distribution of scaling factors had better predictive ability than those models with a mixture prior distribution.

### Items

The following items define specific embodiments of the present invention

Item 1. A method of determining a phenotypic trait in a bovine subject, comprising detecting in a sample from said bovine subject the presence or absence of at least one genetic marker allele or a specific combination of genetic marker alleles, wherein said genetic marker allele or a specific combination of genetic marker alleles is associated with said phenotypic trait of said bovine subject and/or off-spring therefrom.
Item 2. The method according to Item 1, wherein said at least one genetic marker is a single nucleotide polymorphism (SNP), or a microsatellite marker.
Item 3. The method according to any of the preceding, comprising determining multiple genetic marker alleles in a sample from said bovine subject.
Item 4. The method according to Item 3, comprising determining at least 50 genetic marker alleles, such as at least 100 genetic marker alleles, such as at least 200 genetic marker alleles, such as at least 300 genetic marker alleles, such as at least 400 genetic marker alleles, such as at least 500 genetic marker alleles, such as at least 600 genetic marker alleles, such as at least 700 genetic marker alleles, such as at least 800 genetic marker alleles, such as at least 900 genetic marker alleles, such as at least 1000 genetic marker alleles, such as at least 2000 marker alleles, for example at least 3000 marker alleles, such as at least 4000 genetic marker alleles, for example at least 5000 genetic marker alleles, such as at least 6000 marker alleles, for example at least 7000 marker alleles, such as at least 8000 genetic marker alleles, for example at least 9000 genetic marker alleles, such as at least 10000 marker alleles, for example at least 12000 marker alleles, such as at least 14000 genetic marker alleles, for example at least 16000 genetic marker alleles, such as at least 18000 marker alleles, for example at least 20000 marker alleles, for example at least 22000 marker alleles, such as at least 24000 genetic marker alleles, for example at least 26000 genetic marker alleles, such as at least 28000 marker alleles, for example at least 30000 marker alleles, for example at least 32000 marker alleles, such as at least 34000 genetic marker alleles, for example at least 36000 genetic marker alleles, such as at least 38000 marker alleles, for example at least 40000 marker alleles, for example at least 42000 marker alleles, such as at least 44000 genetic marker alleles, for example at least 46000 genetic marker alleles, such as at least 48000 marker alleles, for example at least 50000 marker alleles, for example at least 52000 marker alleles, such as at least 54000 genetic marker alleles, for example at least 56000 genetic marker alleles, such as at least 58000 marker alleles, for example at least 60000 marker alleles, for example at least 62000 marker alleles, such as at least 64000 genetic marker alleles, for example at least 66000 genetic marker alleles, such as at least 68000 marker alleles, for example at least 20000 marker alleles, for example at least 72000 marker alleles, such as at least 74000 genetic marker alleles, for example at least 76000 genetic marker alleles, such as at least 78000 marker alleles, for example at least 80000 marker alleles, for example at least 82000 marker alleles, such as at least 84000 genetic marker alleles, for example at least 86000 genetic marker alleles, such as at least 88000 marker alleles, for example at least 90000 marker alleles, for example at least 92000 marker alleles, such as at least 94000 genetic marker alleles, for example at least 96000 genetic marker alleles, such as at least 98000 marker alleles, for example at least 100000 marker alleles.
Item 5. The method according to any of the preceding, wherein said phenotypic trait is selected from the group consisting of Birth ease, Body score, Calving ease, Fat, Fat percent, Fertility, Health, Leg, Longevity, Milk, Milk organ, Milk speed, Protein, Prot. percent, Temperament, Udder health, Yield, Average, and other diseases.
Item 6. The method according to any of the preceding, wherein said sample is selected from the group consisting of blood, semen (sperm), urine, liver tissue, muscle, skin, hair, follicles, ear, tail, fat, testicular tissue, lung tissue, saliva, spinal cord biopsy, and any other tissue.
Item 7. The method according to any of the preceding, wherein said sample is blood, muscle tissue or liver tissue.
Item 8. The method according to any of the preceding, wherein said sample is blood.
Item 9. The method according to any of the preceding, wherein said bovine subject is a member of the Holstein breed
Item 10. The method according to any of the preceding, wherein said bovine subject is a member of the Danish Holstein cattle population
Item 11. The method according to any of the preceding, wherein at least one of said genetic marker alleles is detected by use of allele specific oligonucleotide primers.
Item 12. A method for selecting bovine subjects for breeding purposes, said method comprising detecting in a sample from said bovine subject the presence or absence of at least one genetic marker allele or a specific combination of genetic marker alleles as defined in any of the preceding, wherein said at least one genetic marker allele or a specific combination of genetic marker alleles is associated with at least one trait according to Item 5 of said bovine subject and/or off-spring therefrom.
Item 13. A diagnostic kit for determining the presence or absence in a bovine subject of at least one genetic marker allele or a specific combination of genetic marker alleles, wherein said genetic marker allele or a specific combination of genetic marker alleles is associated with a phenotypic trait of said bovine subject and/or off-spring therefrom.
Item 14. The diagnostic kit according to Item 13, for determining multiple genetic marker alleles that are associated with a phenotypic trait of said bovine subject and/or off-spring therefrom.
Item 15. The diagnostic kit according to any of Item 13 and Item 14, comprising at least one oligonucleotide for genotyping said bovine subject in respect of a genetic marker allele as defined in Item 1.
Item 16. The kit according to any of Item 13 to Item 15, further comprising reagents and buffers required for genotyping.
Item 17. The diagnostic kit according to Item 16, wherein genotyping is microsatellite genotyping and/or single nucleotide polymorphism genotyping.
Item 18. The diagnostic kit according to any of Item 13 to Item 17 further comprising at least one reference sample.
Item 19. The diagnostic kit according to Item 18, wherein said reference sample comprises at least one oligonucleotide sequence of a genetic marker allele associated with a phenotypic trait as defined in claim.
Item 20. The kit according to any of Item 13 to Item 19, further comprising instructions for the performance of the detection method of the kit, and for the interpretation of the results.

1. A method for determining the individual effect of a plurality of genetic marker alleles on udder health, fertility and/or other health of at least 100 reference bovine subjects and/or its relatives, said method comprising
a. providing at least 100 reference bovine subjects,
b. obtaining a sample from said one or more bovine subjects comprising genetic material,
c. determining on the basis of said genetic material the genotype of said one or more reference bovine subjects for said plurality of genetic markers,
d. determining the udder health, fertility and/or other health of said reference bovine subject, and
e. determining the individual effect of said plurality of genetic marker alleles on said udder health, fertility and/or other health of said reference bovine subject.

2. The method of claim 1, wherein an estimated breeding value (EBV) is calculated on the basis of said udder health, fertility and/or other health.

3. The method of claim 2, wherein the effect of each individual genetic marker allele on udder health, fertility, and/or other health is determined by calculating a reference estimated breeding value (EBV) of said one or more reference bovine subjects, wherein said reference EBV is used as response variable for determining the effect of each individual genetic marker allele on udder health, fertility, other health and/or the EBV.

4. The method of claim 3, wherein said udder health, fertility, other health and/or estimated breeding value is determined by registration of phenotypic traits of said bovine subject and off-spring and/or other relatives of said bovine subject.

5. The method of claim 4, wherein said udder health, fertility, other health and/or estimated breeding value is evaluated by registration of phenotypic traits of at least 40 off-spring or other relatives of said bovine subject.

6. The method of claim 3, wherein the effect of said plurality of genetic markers on udder health, fertility, other health and/or estimated breeding value have been determined for at least 100 reference bovine subjects, such as at least 1000 bovine subjects, for example between 1000 and 6000 bovine subjects, such as between 2000 and 5000 bovine subjects.

7. The method according to claim 3, wherein said udder health, fertility, other health and/or estimated breeding value is determined using Least-squares method, Bayesian estimation, such as BayesA or BayesB or modification thereof, or Best Linear Unbiased Prediction (BLUP), for example marker-assisted Best Linear Unbiased Prediction (MA-BLUP), preferably Bayesian estimation, such as BayesA or BayesB or modifications thereof.

8. The method according to claim 3, wherein said effect of each individual genetic marker allele on the estimated breeding value is determined by Bayesian estimation, such as BayesA or BayesB, or modifications thereof.

9. The method according to claim 3, wherein udder health, fertility, other health, estimated breeding value and/or the individual effect of each genetic marker allele on said breeding value is stored in a non-volatile memory such as a computer memory and/or a database.

10. A method of determining a genomic estimated breeding value (GEBV) of a bovine subject based on the genotype of said bovine subject for a plurality of genetic markers, said method comprising
a. providing a bovine subject,
b. obtaining a sample from said bovine subject comprising genetic material,
c. determining on the basis of said genetic material the genotype of said bovine subject for said plurality of genetic markers,
d. determining said GEBV by correlating said genotype for said plurality of genetic markers with a predetermined effect of each individual genetic marker allele on udder health, fertility, other health and/or estimated breeding value of at least 100 reference bovine subjects, said effect being determined as defined in any one of the preceding claims.

11. The method according to claim 10, wherein said udder health comprises resistance to clinical mastitis.

12. The method according to claim 10, wherein said udder health is determined by an udder health index weighing together information from resistance to clinical mastitis in first, second and/or third parity, somatic cell count (SCC), dairy form, udder support/fore udder attachment, and/or udder depth.

13. The method according to claim 10, wherein said fertility is determined by a fertility index comprising Number of inseminations cows (AISC), Number of inseminations heifers (AISH), Fertility treatment 1st lactation (FERT1), Fertility treatment 2nd lactation (FERT2), Fertility treatments 3rd lactation (FERT3), Heat strength cows (HSTC), Heat strength heifers (HSTH), Calving to first insemination (ICF), First to last insemination cows (IFLC), First to last insemination heifers (IFLH), 56 day Non-return rate cows (NRRC), and/or 56 day Non-return rate heifers (NRRH).

14. The method according to claim 10, wherein said other health is determined by an other health index comprising reproductive diseases, digestive diseases, and/or feet and leg diseases.

15. The method according to claim 10, wherein said plurality of genetic markers or genetic marker alleles is a plurality of single nucleotide polymorphisms (SNP), microsatellite markers and/or mixtures thereof.

16. The method according to claim 15, wherein said plurality of genetic markers or genetic marker alleles comprises at least 50, such as at least 100, such as at least 200, such as at least 300, such as at least 400, such as at least 500, such as at least 600, such as at least 700, such as at least 800, such as at least 900, such as at least 1000, such as at least 2000, for example at least 3000, such as at least 4000, for example at least 5000, such as at least 6000, for example at least 7000, such as at least 8000, for example at least 9000, such as at least 10000, for example at least 12000, such as at least 14000, for example at least 16000, such as at least 18000, for example at least 20000, for example at least 22000, such as at least 24000, for example at least 26000, such as at least 28000, for example at least 30000, for example at least 32000, such as at least 34000, for example at least 36000, such as at least 38000, for example at least 40000, for example at least 42000, such as at least 44000, for example at least 46000, such as at least 48000, for example at least 50000, for example at least 52000, such as at least 54000, for example at least 56000, such as at least 58000, for example at least 60000, for example at least 62000, such as at least 64000, for example at least 66000, such as at least 68000, for example at least 20000, for example at least 72000, such as at least 74000, for example at least 76000, such as at least 78000, for example at least 80000, for example at least 82000, such as at least 84000, for example at least 86000, such as at least 88000, for example at least 90000, for example at least 92000, such as at least 94000, for example at least 96000, such as at least 98000, for example at least 100000 genetic markers or genetic marker alleles.

17. The method according to claim 15, wherein said plurality of genetic markers or genetic marker alleles comprises between 10000 and 100000, such as between 20000 and 80000, for example between 30000 and 60000, for example between 30000 and 50.000, such as between 30000 and 40000, for example between 35000 and 40000, for example between 37000 and 39000 genetic markers or genetic marker alleles.

18. The method according to claim 15, wherein said plurality of genetic markers or genetic marker alleles is selected from the SNP markers of the Illumina Bovine SNP50 BeadChip.

19. (canceled)

20. The method according to claim 15, wherein said bovine subject is a member of the Holstein breed.

21. (canceled)

22. The method according to claim 15, wherein said sample is selected from the group consisting of blood, semen (sperm), urine, liver tissue, milk, muscle, skin, hair, follicles, ear, tail, fat, testicular tissue, lung tissue, saliva, spinal cord biopsy, and any other tissue.

23. (canceled)

24. The method of claim 10, wherein said udder health, fertility, other health and/or genomic estimated breeding value is calculated by simultaneous inclusion of all genetic marker effects regardless of statistic significance, and/or wherein genetic marker effects are calculated simultaneously.

25. The method according to claim 24, wherein said udder health, fertility, other health and/or genomic estimated breeding value is calculated using Least-squares, Bayesian estimation, such as BayesA or BayesB or modifications thereof, or a marker-assisted Best Linear Unbiased Prediction (MA-BLUP), preferably Bayesian estimation, such as BayesA or BayesB or modifications thereof.

26. The method according to claim 25, wherein the genomic estimated breeding value (GEBV) is combined with an estimated breeding value determined on the basis of an observed phenotype of said bovine subject and/or its offspring or other relatives.

27. A method for selective breeding, comprising determining a genomic estimated breeding value (GEBV) of a bovine subject using a method as defined in claim 10, using said bovine subject as sire or dam for breeding if the GEBV of said bovine subject is equal to, or differs less than a predetermined amount from, a desired breeding value for the offspring.

28. The method according to claim 27, wherein the bovine subject is used as sire or dam before an udder health, fertility and/or other health phenotype associated with the genomic estimated breeding value becomes manifest, and/or wherein said bovine subject does not have any off-spring.

29. A computer program product including program code portions for performing, when run on a programmable apparatus, a method according to claim 10.

30. A computer readable medium comprising data representing a computer program product as defined in claim 29.

31. A computer system and/or a programmable apparatus comprising a computer program product as defined in claim 29 and/or computer readable medium comprising data representing a computer program product as defined in claim 29.

32. A kit comprising means for detecting a plurality of genetic markers, said kit comprising a computer program as defined in 29 and/or a computer readable medium comprising data representing a computer program product as defined in claim 29.

33. The kit according to claim 32, further comprising instructions for the performance of the detection method of the kit, and for the interpretation of the results.

34. Use of a computer program product as defined in claim 29 for estimating a breeding value for a specific phenotype of a bovine subject.
